SEQUENCE LISTING (63 sequences)

SEQ ID NO: 1 - DNA, 24 bp, Artificial Sequence
  Description of Artificial Sequence Synthetic primer
gaactctagacacaggacctcacc

SEQ ID NO: 2 - DNA, 30 bp, Artificial Sequence
  Description of Artificial Sequence Synthetic primer
gttcagataattgctaatgctctgacttgc

SEQ ID NO: 3 - DNA, 28 bp, Artificial Sequence
  Description of Artificial Sequence Synthetic primer
agcattagcaattatctgaactggtatc

SEQ ID NO: 4 - DNA, 21 bp, Artificial Sequence
  Description of Artificial Sequence Synthetic primer
tgcgggatccaactgaggaag

SEQ ID NO: 5 - DNA, 30 bp, Artificial Sequence
  Description of Artificial Sequence Synthetic primer
gttcagattattgctaataccctgacttgc

SEQ ID NO: 6 - DNA, 28 bp, Artificial Sequence
  Description of Artificial Sequence Synthetic primer
ggtattagcaataatctgaactggtatc

SEQ ID NO: 7 - DNA, 16 bp, Artificial Sequence
  Description of Artificial Sequence Synthetic primer
acagtcactgagctgg

SEQ ID NO: 8 - DNA, 36 bp, Artificial Sequence
  Description of Artificial Sequence Synthetic primer
gccggatcctcactggatggtgggaagatggataca

SEQ ID NO: 9 - protein, 5 aa, Artificial Sequence
  Description of Artificial Sequence Synthetic peptide
SYNMH

SEQ ID NO: 10 - protein, 7 aa, Artificial Sequence
  Description of Artificial Sequence Synthetic peptide
ATSNLAS

SEQ ID NO: 11 - protein, 5 aa, Artificial Sequence
  Description of Artificial Sequence Synthetic peptide
NYNMH

SEQ ID NO: 12 - protein, 5 aa, Artificial Sequence
  Description of Artificial Sequence Synthetic peptide
KYNMH

SEQ ID NO: 13 - protein, 5 aa, Artificial Sequence
  Description of Artificial Sequence Synthetic peptide
GYNMH

SEQ ID NO: 14 - protein, 5 aa, Artificial Sequence
  Description of Artificial Sequence Synthetic peptide
GYNMH

SEQ ID NO: 15 - protein, 5 aa, Artificial Sequence
  Description of Artificial Sequence Synthetic peptide
GSNMH

SEQ ID NO: 16 - protein, 5 aa, Artificial Sequence
  Description of Artificial Sequence Synthetic peptide
SYNMH

SEQ ID NO: 17 - protein, 5 aa, Artificial Sequence
  Description of Artificial Sequence Synthetic peptide
SYNMH

SEQ ID NO: 18 - protein, 5 aa, Artificial Sequence
  Description of Artificial Sequence Synthetic peptide
SYYMH

SEQ ID NO: 19 - protein, 5 aa, Artificial Sequence
  Description of Artificial Sequence Synthetic peptide
SYNLH

SEQ ID NO: 20 - protein, 5 aa, Artificial Sequence
  Description of Artificial Sequence Synthetic peptide
SFNMQ

SEQ ID NO: 21 - protein, 5 aa, Artificial Sequence
  Description of Artificial Sequence Synthetic peptide
SYNMH

SEQ ID NO: 22 - protein, 7 aa, Artificial Sequence
  Description of Artificial Sequence Synthetic peptide
TTSSLAR

SEQ ID NO: 23 - protein, 7 aa, Artificial Sequence
  Description of Artificial Sequence Synthetic peptide
TTSSLAR

SEQ ID NO: 24 - protein, 7 aa, Artificial Sequence
  Description of Artificial Sequence Synthetic peptide
SKSILAS

SEQ ID NO: 25 - protein, 7 aa, Artificial Sequence
  Description of Artificial Sequence Synthetic peptide
TTSNMAD

SEQ ID NO: 26 - protein, 7 aa, Artificial Sequence
  Description of Artificial Sequence Synthetic peptide
ATSNLAS

SEQ ID NO: 27 - protein, 7 aa, Artificial Sequence
  Description of Artificial Sequence Synthetic peptide
ATPNLDC

SEQ ID NO: 28 - protein, 7 aa, Artificial Sequence
  Description of Artificial Sequence Synthetic peptide
AASNLQS

SEQ ID NO: 29 - DNA, 54 bp, Artificial Sequence
  Description of Artificial Sequence Synthetic primer
gtgcaactgcaggcttccggggctgaggtaaataagcctggggcctcagtgaag

SEQ ID NO: 30 - DNA, 30 bp, Artificial Sequence
  Description of Artificial Sequence Synthetic primer
tacccagtgcatatagtaactggtaaatgt

SEQ ID NO: 31 - DNA, 30 bp, Artificial Sequence
  Description of Artificial Sequence Synthetic primer
tacccagtgcaaattgtaactggtaaatgt

SEQ ID NO: 32 - DNA, 30 bp, Artificial Sequence
  Description of Artificial Sequence Synthetic primer
agttactatatgcactgggtacggcagcct

SEQ ID NO: 33 - DNA, 30 bp, Artificial Sequence
  Description of Artificial Sequence Synthetic primer
agttacaatttgcactgggtacggcagcct

SEQ ID NO: 34 - DNA, 50 bp, Artificial Sequence
  Description of Artificial Sequence Synthetic primer
ggagacggtgaccgtggtgccttggccccagtagtcaaagtagtctacgt

SEQ ID NO: 35 - DNA, 45 bp, Artificial Sequence
  Description of Artificial Sequence Synthetic primer
gatattcagctgacacagtctccatcaagtctttctgcatctgtg

SEQ ID NO: 36 - DNA, 30 bp, Artificial Sequence
  Description of Artificial Sequence Synthetic primer
ggaagccaggatggacttggaataaattac

SEQ ID NO: 37 - DNA, 30 bp, Artificial Sequence
  Description of Artificial Sequence Synthetic primer
atcagccatgttggatgtggtataaattac

SEQ ID NO: 38 - DNA, 30 bp, Artificial Sequence
  Description of Artificial Sequence Synthetic primer
ggactgcaggttggaggcggcataaattac

SEQ ID NO: 39 - DNA, 33 bp, Artificial Sequence
  Description of Artificial Sequence Synthetic primer
tattccaagtccatcctggcttccggagtccct

SEQ ID NO: 40 - DNA, 30 bp, Artificial Sequence
  Description of Artificial Sequence Synthetic primer
accacatccaacatggctgatggagtccct

SEQ ID NO: 41 - DNA, 30 bp, Artificial Sequence
  Description of Artificial Sequence Synthetic primer
gccgcctccaacctgcagtccggagtccct

SEQ ID NO: 42 - DNA, 42 bp, Artificial Sequence
  Description of Artificial Sequence Synthetic primer
ccgtttgatcaccagcttggtcccagcaccgaacgtgagcgg

SEQ ID NO: 43 - DNA, 36 bp, Artificial Sequence
  Description of Artificial Sequence Synthetic primer
gatcggcccagccgggccgtgcaactgcaggcttcc

SEQ ID NO: 44 - DNA, 36 bp, Artificial Sequence
  Description of Artificial Sequence Synthetic primer
gacggtgaccgtggtgccggagacggtgaccgtggt

SEQ ID NO: 45 - DNA, 36 bp, Artificial Sequence
  Description of Artificial Sequence Synthetic primer
gagctcactcagtctccagatattcagctgacacag

SEQ ID NO: 46 - DNA, 36 bp, Artificial Sequence
  Description of Artificial Sequence Synthetic primer
cggcgcacctgcggccgcccgtttgatcaccagctt

SEQ ID NO: 47 - DNA, 108 bp, Artificial Sequence
  Description of Artificial Sequence Synthetic polynucleotide
accacggtcaccgtctcctcaggtggaggcggttcaggcggaggtggctctggcggtggcggatcggacatcgagctcactcagtctccagagctcactcagtctcca

SEQ ID NO: 48 - protein, 107 aa, Artificial Sequence
  Description of Artificial Sequence Synthetic light chain immunoglobin variable region of RFB4
DIQLTQTTSSLSASLGDRVTISCRASQDISNYLNWYQQKPDGTVKLLIYYTSILHSGVPSRFSGSGSGTDYSLTISNLEQEDFATYFCQQGNTLPWTFGGGTKLEIK

SEQ ID NO: 49 - protein, 11 aa, Artificial Sequence
  Description of Artificial Sequence Synthetic peptide
RASQDISNYLN

SEQ ID NO: 50 - protein, 11 aa, Artificial Sequence
  Description of Artificial Sequence Synthetic peptide
RASQSISNYLN

SEQ ID NO: 51 - protein, 15 aa, Artificial Sequence
  Description of Artificial Sequence Synthetic peptide
GGGGSGGGGSGGGGS

SEQ ID NO: 52 - protein, 11 aa, Artificial Sequence
  Description of Artificial Sequence Synthetic peptide
RASQGISNNLN

SEQ ID NO: 53 - DNA, 369 bp, Artificial Sequence
  Description of Artificial Sequence Synthetic heavy chain variable region of IF5
caggtgcaactgcggcagcctggggctgagctggtgaagcctggggcctcagtgaagatgtcctgcaaggcttctggctacacatttaccagttacaatatgcactgggtaaagcagacacctggacagggcctggaatggattggagctatttatccaggaaatggtgatacttcctacaatcagaagttcaaaggcaaggccacattgactgcagacaaatcctccagcacagcctacatgcagctcagcagtctgacatctgaggactctgcggtctattactgtgcaagatcgcactacggtagtaactacgtagactactttgactactggggccaaggcaccactctcacagtctcctctgat

SEQ ID NO: 54 - DNA, 321 bp, Artificial Sequence
  Description of Artificial Sequence Synthetic light chain variable region of IF5
caaattgttctctcccagtctccagcaatcctttctgcatctccaggggagaaggtcacaatgacttgcagggccagctcaagtttaagtttcatgcactggtaccagcagaagccaggatcctcccccaaaccctggatttatgccacatccaacctggcttctggagtccctgctcgcttcagtggcagtgggtctgggacctcttactctctcacaatcagcagagtggaggctgaagatgctgccacttatttctgccatcagtggagtagtaacccgctcacgttcggtgctgggaccaagctggagctcaaacgg

SEQ ID NO: 55 - protein, 123 aa, Artificial Sequence
  Description of Artificial Sequence Synthetic heavy chain variable region of IF5
QVQLRQPGAELVKPGASVKMSCKASGYTFTSYNMHWVKQTPGQGLEWIGAIYPGNGDTSYNQKFKGKATLTADKSSSTAYMQLSSLTSEDSAVYYCARSHYGSNYVDYFDYWGQGTTLTVSSD

SEQ ID NO: 56 - protein, 107 aa, Artificial Sequence
  Description of Artificial Sequence Synthetic light chain variable region of IF5
QIVLSQSPAILSASPGEKVTMTCRASSSLSFMHWYQQKPGSSPKPWIYATSNLASGVPARFSGSGSGTSYSLTISRVEAEDAATYFCHQWSSNPLTFGAGTKLELKR

SEQ ID NO: 57 - protein, 122 aa, Artificial Sequence
  Description of Artificial Sequence Synthetic heavy chain variable region of re-engineered IF5
QVQLVASGAEVNKPGASVKVSCKASGYTFTSYNMHWVRQPPGRGLEWIGAIYPGNGDTSYNQKFKGRVTITADKSTSTAYMELSSLRSEDTAVYYCARSHYGSNYVDYFDYWGQGTTVTVSS

SEQ ID NO: 58 - protein, 107 aa, Artificial Sequence
  Description of Artificial Sequence Synthetic light chain variable region of re-engineered IF5
DIQLTQSPSSLSASVGDRVTITCRASSSLSFMHWYQQKPGQAPVPVIYATSNLASGVPSRFSGSGSGTEFTLTISSLQPEDFATYFCHQWSSNPLTFGAGTKLTVLR

SEQ ID NO: 59 - DNA, 18 bp, Artificial Sequence
  Description of Artificial Sequence Synthetic oligonucleotide
gcggccgcaggtgcgccg

SEQ ID NO: 60 - DNA, 53 bp, Artificial Sequence
  Description of Artificial Sequence Synthetic polynucleotide
gtggcggatcggacatcgagctcactcagtctccagagctcactcagtctcca

SEQ ID NO: 61 - DNA, 40 bp, Artificial Sequence
  Description of Artificial Sequence Synthetic polynucleotide
tcctcaggtggaggcggttcaggcggaggtggctctggcg

SEQ ID NO: 62 - DNA, 18 bp, Artificial Sequence
  Description of Artificial Sequence Synthetic oligonucleotide
ggcaccacggtcaccgtc

SEQ ID NO: 63 - DNA, 17 bp, Artificial Sequence
  Description of Artificial Sequence Synthetic oligonucleotide
gatcggcccagccggcc

(19) United States
(12) Patent Application Publication
(10) Pub. No.: US 2010/0197896 A1
(43) Pub. Date: Aug. 5, 2010

(54) FUNCTIONAL HUMANIZATION OF COMPLEMENTARITY DETERMINING REGIONS (CDRS)

(76) Inventors: Shui-on Leung, Hong Kong (CN); Pui Fan Wong, Hong Kong (CN)

Correspondence Address: SMITH PATENT CONSULTING, LLC, 515 East Braddock Road, Suite B, ALEXANDRIA, VA, 22314, US

(21) Appl. No.: 12/600,307

(22) PCT Filed: May 16, 2008
(86) PCT No.: PCT/US08/63858

§ 371 Date: Apr. 6, 2010

Related U.S. Application Data
(60) Provisional application No. 60/930,371, filed on May 16, 2007.

(51) Int. Cl. C07K 16/00; C12P 21/00

(52) U.S. Cl. 530/387.3; 435/69.6

(57) ABSTRACT

Current humanization approaches for immunoglobulins focus mostly on modifying the framework regions into human sequences. Herein is provided a method for humanizing antibody complementarity-determining regions (CDRs) through functional humanization to reduce the potential immunogenicity of non-human CDR-containing antibodies. CDRs with high sequence homology to the parent CDR are identified from a database of human CDR sequences. One or more human CDRs that are highly homologous to the parent CDR sequence can be used to replace the corresponding CDRs of murine immunoglobulins (or their humanized, or re-engineered versions). Human CDRs that improve or have minimal effects on the antigen binding affinity and specificity are adopted.

### PRIORITY

This application claims the benefit of U.S. Provisional Application Ser. No. 60/930,371 filed May 16, 2007, the entire contents of which are incorporated by reference herein.

### FIELD OF THE INVENTION

The present invention relates to methods of making re-engineered immunoglobulins. More specifically, it relates to replacing the complementarity-determining region (CDR) sequences of a parent immunoglobulin, such as a murine immunoglobulin, with corresponding CDR sequences obtained from a primate or human database. An immunoglobulin thus engineered is considered to be CDR-humanized.

### BACKGROUND

The use of cell fusion to produce monoclonal antibodies from immunized mice described by Kohler and Milstein in 1975 was an important step in the development of antibody technology. Monoclonal antibodies are highly specific and will bind and affect disease-specific targets, thereby sparing normal cells, and presumably causing fewer toxic side-effects than less specific chemical drugs. OKT3, an anti-CD3 murine monoclonal antibody, was the first therapeutic antibody approved by the FDA for uses in the prevention of organ graft rejection in 1986. However, the development of appropriate therapeutic products has been severely hampered due to a number of drawbacks inherent in monoclonal antibodies of murine origin, such as short serum half-life, inability to trigger human effector functions and the production of human anti-mouse antibodies (i.e., the HAMA response).

The advent of other complementary technologies from the 80's through 90's, such as antibody chimerization (see, e.g., U.S. Pat. No. 4,816,567, which is incorporated herein by reference) and humanization (see, e.g. U.S. Pat. Nos. 5,225,539; 5,585,089; 5,693,762; and 5,693,761, which are incorporated herein by reference) of rodent antibodies; phage-display combinatorial libraries (see, e.g., Clackson et al. 1991 Nature 352:624-628; Felici et al. 1991. J Mol. Biol. 222:301-310; and Markland et al. 1991. Gene 109-13-19.), the huMAb/Xeno mouse that produces human antibodies (see, e.g. U.S. Pat. Nos. 6,075,181; 6,150,584; and 7,041,870, which are incorporated herewith by reference), and associated antibody production technologies have eventually revealed the therapeutic potential of monoclonal antibodies. Rituximab, approved in 1997, is the first therapeutic monoclonal antibody used in its naked form for the treatment of cancer; it is now considered the holy grail of antibody-based therapeutics. There are currently more than 23 therapeutic antibodies approved by the US Food and Drug Administration (FDA), and hundreds of antibody candidates being evaluated at various stages of clinical trials.

Primarily, an antibody works by first binding to the target antigen at a unique and specific site. It exerts its therapeutic response either by blocking undesirable interactions with the target cells (see, for example, ReoPro [Abciximab], Remicade [infliximab], and Humira [adalimumab]) or by inducing immune effector functions to eliminate unwanted cells, such as tumors (see, for example, Rituxan [rituximab], Herceptin [trastuzumab], and Campath-1 [alemtuzumab]). Because of its target specificity, others have used the antibody as the delivery vehicle to bring payloads of chemical drugs, such as, for example, Mylotarg [gemtuzumab] or radionuclides, such as, for example, Zevalin [ibritumomab] and Bexxar [tositumomab], to the target cells to effect cytotoxic elimination. The unique target specificities, proven clinical efficacies and safety profiles of therapeutic antibodies have opened up unlimited possibilities for achieving optimal disease diagnosis, therapy, and other industrial applications.

The technical breakthrough that led to the clinical success of therapeutic antibodies comes from the advent of antibody engineering capabilities, such as chimerization and humanization. The technology allows the conversion of most of the murine derived antibodies into human forms without significantly altering the antigen specificity and affinity of the parent antibodies. Chimerization (described in detail in U.S. Pat. No. 4,816,567 cited above) takes the approach of transplanting the heavy and light chain variable regions of the murine antibody onto the human constant region. Therefore, a chimeric antibody contains one third of its sequence in murine form, and in theory, can be immunogenic upon repeated administration into humans. Conventional humanization approaches (described in detail in U.S. Pat. Nos. 5,225,539; 5,585,089; 5,693,762; and 5,693,761 cited above) aim to reduce the percentage murine sequences by grafting the complementarity-determining-region (CDR) sequences of the parent antibody onto acceptor human framework sequences. The resultant humanized antibody usually will contain less than 10% sequence that are of murine origin. This approach is not without deficiencies. First, the CDRs are derived from murine antibodies, and remain to be major sources of “foreignness” (immunogenicity). Secondly, direct grafting of CDRs onto a human framework usually will result in the loss of antibody affinity and specificity, although this loss can be rescued by identifying framework residues that might interact with the antigen binding site and re-introducing these murine residues back onto the human framework; however, it is not uncommon for a CDR-grafted immunoglobulin to contain more than 7 back-mutated residues from the murine frameworks. The major drawback of the conventional CDR-grafting approach is that it only aims to achieve “visual resemblance” to a human antibody, and fails to examine the level of achievable “humanness” from an immunological perspective. It does not take into consideration the possibility of generating novel T-cell immunogenic epitopes by the back-mutated murine residues within the context of the acceptor human frameworks.

In order to avoid employing murine CDRs in the final antibody structure, other techniques have been developed to generate fully human antibodies. Cambridge Antibody Technology (Cambridge, UK) and Dyax Corp. (Cambridge, Mass.) have obtained antibody cDNA sequences from peripheral B cells isolated from immunized humans and devised phage display libraries for the identification of human variable region sequences of a particular specificity. Briefly, the antibody variable region sequences are fused either with the Gene III or Gene VIII structure of the M13 bacteriophage (described in detail in Clackson et al. 1991 Nature 352:624-628; Felici et al. 1991. J Mol. Biol. 222:301-310; and Markland et al. 1991. Gene 109-13-19, cited above). These antibody variable region sequences are expressed either as Fab or single chain Fv (scFv) structure at the tip of the phage carrying the respective sequences. Through rounds of the panning process, using different levels of antigen binding conditions (i.e., stringencies), phages expressing Fab or scFv structures that are specific for the antigen of interest can be selected and isolated. The antibody variable region cDNA sequences of selected phages can then be elucidated using standard sequencing procedures. These sequences may then be used for the reconstruction of a full antibody with the desired isotype using established antibody engineering techniques. Antibodies constructed using this method are considered a fully human antibody (including the CDRs). In order to improve the immunoreactivity (antigen binding affinity and specificity) of the selected antibody, an in vitro maturation process is introduced, including combinatorial association of different heavy and light chains, deletion/addition/mutation at the CDR3 of the heavy and light chains (to mimic V-J, and V-D-J recombination), and random mutations (to mimic somatic hyper-mutation). The anti-Tumor Necrosis Factor a antibody, Humira (adalimumab), is a “fully human” antibody generated by this method that is recently approved by the US FDA for the treatment of Rheumatoid arthritis (RA). The approach suffers from the limitation of lacking sequence diversity as all sequences are derived originally from existing antibodies in the human from whom matured B cells are obtained. Moreover, the introduced mutations in the in vitro maturation process can be potential sources of foreign-ness (new T-cell epitopes), raising questions on the claimed humanness of the phage-library derived antibodies. In fact, repetitively dosed Humira (adalimumab) showed anti-antibody response (AAR) in 12% of patients, raising questions on the extent of humanness achievable by the approach (Abbott. USA. Adalimumab Product Approval Information. 2003).

Perhaps, mice carrying human genomic immunoglobulin gene sequences generated through a series of gene knock-out and transgenic processes (see, e.g. U.S. Pat. No. 6,075,181 (Abgenix); U.S. Pat. No. 6,150,584 (Abgenix); and U.S. Pat. No. 7,041,870 (Medarex), which are incorporated herewith by reference) represent the best source for producing fully human antibodies. These mice (namely, the XenoMouse of Abgenix Inc., Fremont, Calif.; and the HuMAb Mouse of GenPharm-Medarex, San Jose, Calif.) can be immunized with the antigen of interest, and the antibody affinity maturation process is accomplished in a natural immune environment. Although the V, J gene segments for the light chain, and the V, D, J gene segments for the heavy chain are 100% of human origin, the mutation/deletion/addition in the VJ and VDJ junction, and the somatic mutations along the variable region sequence occurring under the murine immune system might differ significantly from that of human. One cannot rule out the possibility of these mutations being potential sources of T-cell epitopes under the human immune surveillance. In fact, the anti-CD20 antibody (HuMax-CD20) derived from the HuMAb Mouse of GenPharm-Medarex (San Jose, Calif.) was demonstrated to be more immunogenic than Rituximab (chimeric anti-CD20) by eliciting higher incidences of infusion reactions in patients with Rheumatoid Arthritis (see Editorial Comment in abstract P0018. Ostergaard et al. 2006. First Clinical Results of Humax-CD20 Fully Human Monoclonal IgG1 Antibody Treatment in Rheumatoid Arthritis (RA). EULAR). Moreover, due to the limited size of the immunoglobulin minigene introduced in the transgenic mice, the diversity generated may not compete with that of the natural human immune system. Regardless, antibodies generated from these mice are considered the most human-like when compared to those generated by other methods.

Except for the HuMab mouse approach, most methods dealing with antibody humanness do so from a visual but not functional perspective. Making the antibody resemble a human antibody in appearance has become the primary goal. However, from a functional perspective, the immunoglobulin protein is in fact examined and inspected by the immune system. An immunoglobulin that gets internalized into an antigen presenting cell (APC) will be proteolytically degraded into linear stretches of peptides. Some resulting peptide fragments are bound to major histocompatibility complex (MHC) class II molecules. A small number of those peptides are expressed on the cell surface as a complex with MHC molecules. Those MHC-peptide complexes evoke an effector response when recognized by the antigen-specific receptors on T cells. This triggers a cascade in which some T cells differentiate into helper T cells. The release of cytokines induces differentiation of antigen-specific B cells into antibody-specific plasma cells. Only when an immunoglobulin contains peptides viewed by the immune system to be “self” will the immunoglobulin be considered fully human and survive the immune surveillance.

Conventional humanization methods that utilize CDR-grafting cannot get rid of the murine sequence of the CDRs which are important for the antigen binding specificity and affinity of the re-engineered antibody. Moreover, the back-mutation required in most CDR-grafting approach may introduce new T-cell epitopes, leading to potential immunogenicity of the CDR-grafted antibodies. Although framework-patching (or framework-re-engineering) technology has mitigated or avoided the need for back-mutation (see, for example, U.S. Pat. Nos. 7,321,026 and 7,338,659, which are incorporated by reference herein), the problem of inherent immunogenicity arising from the CDRs has not been fully resolved.

Other attempts have been made to mitigate the potential immunogenicities imparted by the murine CDRs during antibody humanization. For example, by using a technique called SDR grafting (see, e.g., Gonzales et al. 2003. Minimizing immunogenicity of the SDR-grafted humanized antibody CC49 by genetic manipulation of the framework residues. Mol. Immunol. 40:337-349; Gonzales et al. 2004. SDR grafting of a murine antibody using multiple human germline templates to minimize its immunogenicity. Mol. Immunol. 41:863-872; Kashmiri et al. 2005. SDR-grafting—a new approach to antibody humanization. Methods 36:25-34, which are incorporated by reference herein), amino acid residues within the CDR region that are determined to be important for making contacts with the antigen are identified, and retained as in the murine CDR while the rest of the “less important residues” within the CDR region will be adopted directly from the human immunoglobulin from which the framework is chosen as the acceptor sequence for SDR grafting. Again, the problem of immunogenicity is being dealt with from a visual perspective, and effort is made to minimize the number of murine residues in the final re-engineered immunoglobulin. Yet, the approach suffers from the same problems as in conventional CDR-grafting in which the back-mutation introduced in the framework for immunoreactivity restoration can itself be potential source of immunogenicity as they may be generating new T cell epitopes. Murine residues appearing on a stretch of human sequence will constitute novel sequences new to the human immune surveillance system.

Gillies et al. (e.g. see U.S. Pat. No. 6,992,174, which is incorporated herewith by reference) argues that if a protein sequence does not contain linear peptide sequence that will be presented by the host antigen presenting cells in the context of MHC II as foreign (T-cell epitope), such protein sequence will be “viewed” by the host immune system as “self,” and can be used repeatedly in the host for therapeutic purposes with substantially mitigated risks of eliciting an unfavorable immune response against the protein. They therefore developed a procedure called “peptide threading” process (computational methods based on modeling peptide binding to MHC Class II molecules), with the assistance of computers, to identify stretches of potentially immunogenic peptides that can be presented by the MHC II as foreign, and convert the sequence (usually by changing one or two amino acids) into one that will not be presented as foreign (i.e. unable to bind to MHC Class Il of the APC) by the immune system. By doing these exercises, any highly immunogenic protein (including murine immunoglobulins) with therapeutic potential can in theory be rendered non-immunogenic (deimmunized) by a few mutations in the amino acid sequences (see, e.g., Adair F. 2000. Immunogenicity: The last hurdle for clinically successful therapeutic antibodies. BioPharm 13: 42-46; Adair et al. 2002. The immunogenicity of therapeutic proteins. BioPharm February issue, p 30-36, incorporated by reference herein). This technique/approach requires a thorough understanding of the sequence requirements for a peptide to be rendered immunogenic or non-immunogenic for presentation by the MHC, and the availability of a properly designed computer program for evaluating these sequences.

A review of the crystal structure determinations of highly specific antibody fragments (Fab and Fv) complexed to protein antigens reveals a general consensus on these points: (1) both the L and H chains of antibodies make significant contacts with antigen, although frequently those made by the H chain are more extensive; (2) the specificity of binding is determined, mostly if not totally, by the complementarity determining regions (CDR) of VH and VL; often the VH CDR3 encoded by the D(diversity) gene segment makes important contributions to binding; (3) the contacting residues of the antigen are discontinuous in sequence but form a contiguous surface (antigen determinant or epitope); (4) the contacting surfaces of the antibody and antigen show a high degree of complementarity; (5) these surface areas of interaction are about 600 to 900 Å; (6) van der Waals interactions, hydrogen bonds, and, to a much lesser extent, salt bridges mediate the binding of antibody to antigen; (7) a large proportion of CDR aromatic residues is implicated in the contacts with antigen (Reviewed by Bradford et al. 1995. Structural features of the reactions between antibodies and protein antigens. FASEB J. 9:9-16).

Not all the CDR residues of a murine antibody (or antibodies from other species) are essential for antigen binding. Based on the comprehensive analysis of the three-dimensional structures of the antibody combining sites, it has been suggested that only 20-33% of CDR residues are critical in the antigen-antibody interaction (see, e.g., Padlan E A. 1994. Mol. Immunol. 31:169-217). In an attempt to study the effect of somatic mutations on the binding affinity and specificity of antibodies, Chen et al. (EMBO 14:278402749, 1995) introduced random mutations at the CDR2 of the VH of two anti-phosphocholine antibodies, T15 and D16 (they shared identical VH CDR2 sequence). Out of 43 mutations introduced, 17 and 22 resulted in the complete loss; and 7 and 4 in the reduction of immunoreactivities for T15 and D16, respectively. However, 19 and 10 of the mutations at the VH CDR2 of T15 and D16 did not alter the antigen binding affinity of the antibodies. In the case of D16, 7 of the mutations introduced had actually enhanced the affinity of the antibody (Chen et al. 1995. Enhancement and destruction of antibody function by somatic mutations: unequal occurrence is controlled by V gene combinatorial associations. EMBO 14:2784-2794). In mutation M257, which contained 4 non-conservative point mutations at the VH CDR2 (19 amino acids), both the T15 and D16 exhibited preserved immunoreactivities (up to 20% changes in sequence).

Another study on the binding of the monoclonal antibody NC41 to the viral antigen, influenza virus N9 sialidase showed that only five of the six CDRs actually made contact with the sialidase antigen; the light chain CDR1 does not make contact with the antigen. (Tulip et al., 1991. Refined atomic structure of N9 subtype influenza virus neuraminidase and escape mutants. J. Mol. Biol. 221:487-497).

Accordingly, in light of the foregoing, there remains a need in the art for improved antibody humanization procedures and resulting improved humanized antibodies having reduced immunogenicity. The present invention addresses this need.

### SUMMARY OF THE INVENTION

It was herein discovered that the immunogenicity of a murine antibody can be further reduced above all available humanization methods by independently humanizing each individual CDR of the parent immunoglobulin. It will be understood by those skilled in the art that one or more aspects of this invention can meet certain objectives, while one or more other aspects can meet certain other objectives. Each objective may not apply equally, in all its respects, to every aspect of this invention. As such, the following objects should be viewed in the alternative with respect to any one aspect of this invention.

Thus, in one aspect, the present invention provides a re-engineered immunoglobulin having at least one complementarity-determining region (CDR) whose amino acid sequence is replaced with the amino acid sequence of the corresponding CDR of a primate immunoglobulin. An immunoglobulin so engineered binds to an antigen with an affinity within 50-fold, 30-fold, 20-fold, 10-fold, 5-fold, 3-fold, or 2-fold of the affinity of the parent immunoglobulin for the same antigen. In certain embodiments, the primate CDR has an amino acid sequence that is at least 50% identical to the amino acid sequence of the replaced parent CDR. In other embodiments, the primate CDR contains at least one identical aromatic amino acid residue at the corresponding position of the parent CDR. In still other embodiments, the primate CDR contains at least one identical charged amino acid residue at the corresponding position of the parent CDR. In yet other embodiments, the primate CDR contains at least one amino acid residue identical to the parent CDR at a position that is determined by crystal structure and/or computer analysis to contribute to maintaining the binding affinity of the re-engineered immunoglobulin within 50-fold, 30-fold, 20-fold, 10-fold, 5-fold, 3-fold, or 2-fold of the affinity of the parent immunoglobulin for the same antigen. In a preferred embodiment, the primate species is human.

In another aspect, the present invention provides a method of selecting a CDR from a mammalian species to replace the corresponding CDR of the parent immunoglobulin. The method includes (a) providing the amino acid sequence of a non-human parent immunoglobulin that binds to an antigen; and (b) identifying at least one primate CDR whose amino acid sequence is homologous to the amino acid sequence of a CDR of the parent immunoglobulin.

In a further aspect, the invention provides a method of preparing a re-engineered immunoglobulin. The method includes the steps of (a) providing the amino acid sequence of a non-human parent immunoglobulin that binds to an antigen, and (b) identifying at least one primate CDR whose amino acid sequence is homologous to the amino acid sequence of a CDR of the parent immunoglobulin (steps (a) and (b) above). The method further includes the steps of (c) replacing the parent CDR with the primate CDR in the parent immunoglobulin amino acid sequence; (d) preparing a nucleic acid sequence that encodes the amino acid sequence obtained in step c; and (e) expressing the nucleic acid sequence obtained in step d in a recombinant cell to obtain the re-engineered immunoglobulin. An immunoglobulin so engineered binds to the antigen with an affinity within 50-fold, 30-fold, 20-fold, 10-fold, 5-fold, 3-fold, or 2-fold of the affinity of the parent immunoglobulin for the same antigen.

According to the present invention, a variety of primate (e.g., human) immunoglobulin sequence databases are provided. Separate databases in tangible form are provided for each of the immunoglobulin light chain CDR1, CDR2 and CDR3 segments as well as for each of the immunoglobulin heavy chain CDR1, CDR2, and CDR3 segments.

In one aspect, the present invention provides a database of immunoglobulin light chain variable region sequences in tangible form, i.e., on a storage medium, such as an electronic, magnetic, or optical storage medium, or in printed form. The database contains the amino acid sequences, or nucleotide sequences encoding such amino acid sequences, of at least 2, 5, 10, 20, 50, 100, 200, 500, 1000, 2000, 5000, or 10000 light chain variable regions of a single mammalian species. In a preferred embodiment, the species is human. In some embodiments, the database contains sequences of only kappa chains. In other embodiments, the database contains sequences of only lambda chains. In still other embodiments, the database contains sequences obtained from both kappa and lambda chains.

In yet a further aspect, the present invention provides a DNA library containing DNA sequences encoding at least 2, 5, 10, 20, 50, 100, 200, 500, 1000, 2000, 5000, or 10000 light chain variable region amino acid sequences of the previously described immunoglobulin light chain variable region sequence database.

In another aspect, the invention provides a database of immunoglobulin heavy chain variable regions sequences in tangible form, i.e., on a storage medium or in printed form. The database contains the amino acid sequences, or nucleotide sequences encoding such amino acid sequences, of at least 2, 5, 10, 20, 50, 100, 200, 500, 1000, 2000, 5000, or 10000 heavy chain variable regions of a single mammalian species. In a preferred embodiment, the species is human. In some embodiments, the database contains sequences of only gamma chains. In other embodiments, the database contains sequences of other types (e.g., γ1, γ2, γ3, γ4, μ, α, α2, δ, or ε) of heavy chains. In still other embodiments, the database contains sequences obtained from any possible mixture of the above mentioned heavy chain types.

In further aspect, the present invention provides a DNA library containing DNA sequences encoding at least 2, 5, 10, 20, 50, 100, 200, 500, 1000, 2000, 5000, or 10000 heavy chain variable region amino acid sequences of the previously described immunoglobulin heavy chain variable region sequence database.

In yet another aspect, the present invention provides a phage display library containing scFv or Fab of the parent immunoglobulin in which one or more of its CDR(s) is/are replaced by the CDR(s) selected from a mammalian species based on the aforementioned method. In a preferred embodiment, the species is human.

In yet another aspect, the present invention is a method to maximize the number of CDRs of the parent immunoglobulin that can be replaced by the corresponding CDRs of a mammalian species without significantly affecting the specificity and affinity of the resultant immunoglobulin. In one embodiment, the method involves the introduction of mutations at the heavy chain CDR3. Yet in another embodiment, the method involves the introduction of mutations at the light chain CDR3. In still other embodiments, the method involves the introduction of mutations at the CDR3 of both the heavy and light chain.

These and other objects and features of the invention will become more fully apparent when the following detailed description is read in conjunction with the accompanying figures and/or examples. However, it is to be understood that both the foregoing summary of the invention and the following detailed description are of preferred embodiments and not restrictive of the invention or other alternate embodiments of the invention. In particular, while the invention is described herein with reference to a number of specific embodiments, it will be appreciated that the description is illustrative of the invention and is not constructed as limiting of the invention. Various modifications and applications may occur to those who are skilled in the art, without departing from the spirit and the scope of the invention, as described by the appended claims. Likewise, other objects, features, benefits and advantages of the present invention will be apparent from this summary and certain embodiments described below, and will be readily apparent to those skilled in the art having knowledge of antibody design and antibody library construction. Such objects, features, benefits and advantages will be apparent from the above in conjunction with the accompanying examples, data, figures and all reasonable inferences to be drawn there-from, alone or with consideration of the references incorporated herein.

### BRIEF DESCRIPTION OF THE DRAWINGS

Other features and advantages of the present invention will be apparent from the following detailed description of the invention, taken in conjunction with the accompanying drawings of which:

FIG. 1 provides the amino acid sequence of the light chain immunoglobulin variable region of RFB4 (SEQ ID NO: 48). CDRs are underlined. FIG. 2 provides an amino acid sequence comparison of cRFB4 L1 with the most homologous human L1 (SEQ ID NOS 49, 50 & 52 are disclosed respectively in order of appearance).

FIG. 3 provides the DNA sequences of the heavy (V_{H}) (FIG. 3A) (SEQ ID NO: 53) and light chain (V_{K}) (FIG. 3B) (SEQ ID NO: 54) variable regions of 1F5 antibody. FIG. 4 provides the amino acid sequences of the heavy (V_{H}) (FIG. 4A) (SEQ ID NO: 55) and light chain (V_{K}) (FIG. 4B) (SEQ ID NO: 56) variable regions of 1F5 antibody. CDR regions are boxed.

FIG. 5 provides the amino acid sequences of the heavy (V_{H}) (FIG. 5A) (SEQ ID NO: 57) and light chain (V_{K}) (FIG. 5B) (SEQ ID NO: 58) variable regions of framework re-engineered 1F5 antibody (fr1F5). CDR regions are boxed.

FIG. 6 provides the results of competition flow cytometry of murine 1F5 and fr1F5 (framework-re-engineered) antibody against FITC-conjugated-fr1F5 antibody

FIG. 7 provides an illustration of the primers and PCR-procedures required for joining the VH and VK sequence via the (GGGGS)₃ (SEQ ID NO: 51) linker (SEQ ID NOS 59-63 are disclosed respectively in order of appearance).

FIG. 8 provides the results of binding assays with scFv-Phage containing different CDR-humanized sequences on Raji cell surface antigen extracts.

### DETAILED DESCRIPTION OF THE INVENTION

This present invention constitutes a marked improvement in the production of humanized antibodies. In particular, the present invention provides re-engineered CDR-humanized immunoglobulin in which the complementarity-determining region (CDR) sequences of a parent immunoglobulin, such as a murine immunoglobulin, have been replaced with corresponding CDR sequences obtained from a primate or human database.

Although any methods and materials similar or equivalent to those described herein can be used in the practice or testing of embodiments of the present invention, the preferred methods, devices, and materials are now described. However, before the present materials and methods are described, it is to be understood that this invention is not limited to the particular compositions, methodologies or protocols herein described, as these may vary in accordance with routine experimentation and optimization. It is also to be understood that the terminology used in the description is for the purpose of describing the particular versions or embodiments only, and is not intended to limit the scope of the present invention which will be limited only by the appended claims.

Unless otherwise defined, all technical and scientific terms used herein have the same meaning as commonly understood by one of ordinary skill in the art to which this invention belongs. In case of conflict, the present specification will control.

In the context of the present invention, the words “a”, “an”, and “the” as used herein mean “at least one” unless otherwise specifically indicated.

As used herein, an “immunoglobulin” refers to a protein consisting of one or more polypeptides substantially encoded by immunoglobulin genes. A typical immunoglobulin protein contains two heavy chains paired with two light chains. A full-length immunoglobulin heavy chain is about 50 kD in size (approximately 446 amino acids in length), and is encoded by a heavy chain variable region gene (about 116 amino acids) and a constant region gene. There are different constant region genes encoding heavy chain constant region of different isotypes such as alpha, gamma (IgG1, IgG2, IgG3, IgG4), delta, epsilon, and mu sequences. A full-length immunoglobulin light chain is about 25 Kd in size (approximately 214 amino acids in length), and is encoded by a light chain variable region gene (about 110 amino acids) and a kappa or lambda constant region gene. Naturally occurring immunoglobulin is known as antibody, usually in the form of a tetramer consisting of two identical pairs of immunoglobulin chains, each pair having one light and one heavy chain. In each pair, the light and heavy chain variable regions are together responsible for binding to an antigen, and the constant regions are responsible for the effector functions typical of an antibody.

The present invention relates to humanized immunoglobulin. In the context of the present invention, a humanized antibody will have to have the following characteristics:
(1) significantly reduced, more preferably eliminated immunogenicity, thereby allowing multiple injection of the antibody for human uses;
(2) minimally perturbed immunoreactivity including specificity and affinity (within 3-fold) against the original antigen;
(3) capable of inducing human effector functions such as complement fixation, complement-mediated cytotoxicity, antibody-dependent cell cytotoxicity, etc.

Antibody immunogenicity may be routinely assayed using conventional technology, typically in a clinical setting using suitable subjects, for example, primates, more preferably humans. For example, the immunogenic potential of a therapeutic antibody of interest can be determined by identifying specific T cell epitopes that arise in response to administration the antibody of interest or by determining the potential of normochromatic erythrocytes (NCEs) to stimulate helper T cell responses and/or induce late onset allergy like reactions in response thereto. This process may be automated, for example using the EpiScreen™ technology commercially available through Antitope Ltd (Cambridge UK).

The functionally humanized and presumably non-immunogenic immunoglobulins of the present invention will typically find use individually, or in combination with other treatment modalities, in treating diseases susceptible to antibody-based therapy. For example, the immunoglobulins can be used for passive immunization, or the removal of unwanted cells or antigens, such as by complement mediated lysis, all without substantial adverse immune reactions (for example anaphylactic shock) associated with many prior antibodies. Alternatively, the immunoglobulins of the present invention may be used for in vitro purposes, for example, as diagnostic tools for the detection of specific antigens, or the like.

A preferable usage of the immunoglobulins of the present invention will be the treatment of diseases using their naked forms (naked antibodies) at dosages ranging from 50 mg to 400 mg/m², administered either locally at the lesion site, subcutaneously, intravenously, and intramuscularly, etc. Multiple dosing at different intervals will be performed to achieve optimal therapeutic or diagnostic responses, for example, at weekly intervals, once a week, for four weeks. Usage of the immunoglobulins derived from the present invention can be combined with different treatment modalities, such as chemotherapeutic drugs (for example CHOP, Do, 5-Fu, . . . etc), radiotherapy, radioimmunotherapy, vaccines, enzymes, toxins/immunotoxins, or other immunoglobulins derived from the present invention or others. For example, if the immunoglobulins of the present invention are specific for the idiotype of an anti-tumor antibody, it may find utility can as a tumor vaccine for the elicitation of Ab3 against a tumor antigen. Numerous additional agents, or combinations of agents, well-known to those skilled in the art may also be utilized.

Additionally, the immunoglobulins of the present invention can be utilized in different pharmaceutical compositions. The immunoglobulins can be used in their naked forms, or as conjugated proteins with drugs, radionuclides, toxins, cytokines, soluble factors, hormones, enzymes (for example carboxylesterase, ribonuclease), peptides, antigens (as tumor vaccine), DNA, RNA, or any other effector molecules having a specific therapeutic function with the antibody moiety serving as the targeting agents or delivery vehicles. Moreover, the immunoglobulins or immunoglobulin derivatives, such as antibody fragments, single-chain Fv, diabodies, etc. of the present invention can be used as fusion proteins to other functional moieties, such as, immunoglobulins or immunoglobulin derivatives of a different invention (for example as bispecific antibodies), toxins, cytokines, soluble factors, hormones, enzymes, peptides, etc. Different combinations of pharmaceutical composition, well-known to those skilled in the art may also be utilized.

The materials and methods of the present invention may be utilized to screen for antibodies having binding specificity for a target antigen interest. As noted previously, the humanized immunoglobulins of the present invention may have diagnostic and/or therapeutic utility. Accordingly, the present invention is not limited in terms of the antigen of interest. Examples of antigens of interest suitable for use in the context of the present invention include, but are not limited to, the CD41 7E3 glycoprotein IIb/IIIa receptor on the platelet membrane (associated with cardiovascular disease), TNF (associated with inflammatory conditions), CD52 (associated with chronic lymphocytic leukemia), IL-2a (associated with transplant rejection), VEGF (associated with macular degeneration and colorectal cancer), EGF (associated with colorectal cancer), complement system protein C5 (associated with inflammatory conditions), CD3 receptor (associated with transplant rejection), T cell VLA4 receptor (associated with autoimmune-related multiple sclerosis), CD11a (associated with inflammatory conditions such as psoriasis), CD20, CD22, CD19, Invariant Chain Ii (associated with non-Hodgkins lymphoma and possibly autoimmune diseases), CD33 (associated with acute myelogenous leukemia), IgE inflammatory (associated with allergy-related asthma therapy), the F protein of RSV (associated with RSV), ErbB2 (associated with breast cancer), CEA (associated with colorectal cancer, breast cancer and a variety of tumors), Mucin (associated with breast cancer, pancreatic cancer), CD147 (associated with liver cancer), and beta-amyloid protein (associated with Alzheimer's disease).

The ability of an expressed immunoglobulin to bind a target antigen of interest may be assayed using conventional technology, for example, direct or competition cell binding assays (e.g., cell-based ELISA and/or flow cytometry), ELISA assays (e.g., wherein ELISA plates are coated with the antigen of interest and binding of the antibody directly on to the antigen coated plates is measured using colorimetric methods), Biacor assays (e.g., measuring the affinity of an antibody to a particular antigen), and the like.

The diagnostic and/or therapeutic utility of an immunoglobulin of the present invention may be assayed and confirmed using conventional technology, for example, through the elicitation of complement-mediated cytolysis (CMC), or Antibody Dependent Cell Cytotoxicity (ADCC) on cells expressing the antigen of interests, or by blocking the activity of a particular enzyme or functional protein (for example, blocking cell proliferations of interleukine dependent cell lines with antibodies specific for a particular interleukin).

The present invention makes reference to amino acids and/or charged residues that are “identical” or “conservatively similar”. In the context of the present invention, the term “conservatively similar” refers to the art-recognized process of conservative substitution of amino acids having similar properties. In the process of conservative substitution, an amino acid residue to be mutated is preferably mutated into a different amino acid in which the properties of the amino acid side-chain are conserved. Accordingly, conservative substitutions are expected to exert little to no effect on the activity of the resulting protein. Examples of amino acids grouped by side chain property include hydrophobic amino acids (alanine, isoleucine, leucine, methionine, phenylalanine, proline, tryptophan, tyrosine, valine), hydrophilic amino acids (arginine, aspartic acid, aspargine, cystein, glutamic acid, glutamine, glycine, histitidine, lysine, serine, threonine), and side chains having the following functional groups or characteristics in common: an aliphatic side-chain (glycine, alanine, valine, leucine, isoleucine, praline); a hydroxyl group containing side-chain (serine, threonine, tyrosine); a sulfur atom containing side-chain (C, M); a carboxylic acid and amide containing side-chain (aspartic acid, aspargine, glutamic acid, glutamine); a base containing side-chain (arginine, lysine, histidine); and an aromatic containing side-chain (histidine, phenylalanine, tyrosine, tryptophan). Furthermore, conservative substitution tables providing functionally similar amino acids are well known in the art. For example, the following eight groups each contain amino acids that are conservative substitutions for one another:
1) Alanine (A), Glycine (G);
2) Aspartic acid (D), Glutamic acid (E);
3) Aspargine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cystein (C), Methionine (M) (see, e.g., Creighton, Proteins 1984).

The present invention is based on two concepts: (1) there is room for sequence modifications within each CDR regions without significant effects on the immunoreactivity of the resultant immunoglobulin; (2) a human CDR selected for replacing the corresponding non-human (e.g., murine) parent CDR has been screened by the human immune system to be “self”, and the resultant immunoglobulin will be a less immunogenic protein when compared with the one without the CDR replacement (CDR-humanization).

Given a sufficiently large database for human immunoglobulin V region sequences, human CDR sequences can be identified that exhibit high homology with CDR sequences of parent immunoglobulins. Therefore, human CDR sequences can be used to replace those of parent immunoglobulins without significantly altering their resultant immunoreactivities. Parent immunoglobulin (e.g., murine) CDRs are identified according to the classification of the Kabat Database as H1 (CDR1), H2 (CDR2) and H3 (CDR3) for VH; and L1 (CDR1), L2 (CDR2) and L3 (CDR3) for VL. The individual CDR sequences can be humanized by comparing their sequences with the human database, and human CDR sequences that satisfy one or more, or all, of the following criteria can be selected for CDR humanization:
1. exhibit 50% or higher sequence homology to the corresponding parent CDR;
2. contain aromatic residues identical to the parent CDR at corresponding positions;
3. contain charged residues identical to the parent CDR at corresponding positions; and/or
   contain amino acid residues identical to the parent CDR at positions that are known to be important for maintaining the binding site structure/contacts of the immunoglobulin as determined by crystal structure and/or computer database analysis.

If human or primate CDRs that fulfill these criteria are not available, CDR sequences that have the following characteristics can be used:
1. exhibit 50% or higher sequence homology to the corresponding parent CDR;
2. contain at least one identical aromatic residues at positions corresponding to the parent CDRs;
3. contain at least one identical charged residues at positions corresponding to the parent CDRs; and/or
   contain at least one identical amino acid residues within the CDRs at positions that are known to be important for maintaining the binding site structure/contacts of the immunoglobulin as determined by crystal structure and/or computer database analysis.

Alternatively, human or primate CDR sequences with the following characteristics can be adopted:
1. exhibit 50% or higher sequence homology to the corresponding parent CDR;

12. contain aromatic residues that are identical and/or conservatively similar to the parent CDR at corresponding positions;
3. contain charged residues that are identical and/or conservatively similar to the parent CDR at corresponding positions; and/or
4. contain amino acid residues that are identical and/or conservatively similar to the parent CDR at positions that are known to be important for maintaining the binding site structure/contacts of the immunoglobulin as determined by crystal structure and/or computer database analysis.

If all of the above fail, human or primate CDR sequences with the following characteristics can be used:
1. exhibit 50% or higher sequence homology to the corresponding parent CDR;
2. contain at least one conservatively similar aromatic residues at positions corresponding to the parent CDRs;
3. contain at least one conservatively charged residue at positions corresponding to the parent CDRs; and/or
4. contain at least one conservatively similar amino acid residues at positions corresponding to the parent CDRs that are known to be important for maintaining the binding site structure/contacts of the immunoglobulin as determined by crystal structure and/or computer database analysis.

While methods to identify the percent sequence homology, or percent sequence identity are well known in the art, and the presence of aromatic and charged residues within the CDR sequences is straightforward, identifying CDR residues that are important in maintaining the binding site structure or antibody-antigen contacts requires the use of available databases; crystal structure analysis, and/or computer modeling studies.

When the three-dimensional structure of an antigen-antibody complex is determined based on X-ray crystallographic studies, the residues of the combining site directly involved in ligand contact can be easily identified. In the absence of a three-dimensional structure, CDR residues important for maintaining the binding site structure and antigen contact surfaces can be identified by modeling, by correlating with known sequence variability, or by examining the known crystal structures of antibody-ligand complexes that are currently available in a database, such as the PDB database (Padlan et al. 1995. FASEB J. 9:133-139). An analysis of antibody-ligand complexes of known structure using a database can determine the occurrence rate of a residue at a particular position along a CDR that would be buried, partly buried, exposed, or directly involved in ligand binding. Packing interactions between the antibody and its antigen can be modeled, and this can be one basis for selecting a donor CDR sequence to replace a parent CDR sequence. For example, packing interactions between the parent antibody and the antigen which it binds are preferably preserved in a re-engineered immunoglobulin according to the invention. Packing interactions, which can be set, for example, equal to a 200% van der Waals surface, can be determined as described in Winter U.S. Pat. No. 6,548,640 (hereby incorporated by reference). An effort is made to select human CDRs that retain identical or conservatively similar residues at positions with high chances of being exposed or involved in ligand binding.

As the database for human CDR sequence grows in size, the chances of finding a stretch of CDR sequence fulfilling the above criteria to replace the corresponding murine CDR without negatively affecting the immunoreactivity of the resultant immunoglobulin will be significantly enhanced. When the CDR sequence is of human origin, it should have already been “screened” by the human immune system to be “self”. Therefore, when the immunoglobulin containing this stretch of CDR sequence is internalized and proteolytically degraded within an Antigen Presenting Cell, peptide derived from the human CDR will be presented by the APC in the context of MHC II as self. The approach of the present invention utilizes the human immune system as a high through-put screening system to provide stretches of CDR sequences approved by the screening process as being “self”. That will save the employment of complicated crystal structure analyses and uses of sophisticated programs such as peptide threading to eliminate potential T cell epitope. Using the above criteria, it is possible that a collection of appropriate human CDRs can be used to replace some, if not all, of the original parent non-human CDRs. This can be achieved with the following strategies:

1. Sequential Replacement of Parent CDRs with Human CDRs:

Generally speaking, the CDRs of heavy chain contributes more to antigen-binding than that of light chain; within each immunoglobulin chain, the order of importance is CDR3>CDR2>CDR1. Thus, in one embodiment of the invention, the process of humanizing the CDRs of a non-human antibody is practiced in the following order: L1→H1→L2→H2→L3→H3. For example, the three to ten human L1 from the database that best fit the above criteria can be used to replace the corresponding L1 of the murine immunoglobulin using standard techniques in molecular biology. Antibody variants carrying different human L1 sequences can be expressed, and antigen specificity and affinity tested. The human L1 sequence that gives rise to the highest antigen binding antibody can be chosen. The same procedure can be used for the identification and examination of the best human H1 sequence, then the best L2 sequence, the best H2, the best L3, and finally the best H3.

In an alternative embodiment, one can start with the shortest CDR (except CDR3, which plays a more critical role in determining immunoreactivity). Thus, in most cases, using this strategy the order of CDR replacement can be H1→L2→L1→H2→L3→H3. Variations in the order are also within the scope of the invention.

To facilitate the testing of different combinations of substituted CDRs, one can employ phage-display library techniques and/or other similar technologies such as ribosome-display technology. Briefly, the antibody variable region sequences containing different combinations of human CDRs are fused either with the Gene III or Gene VIII structure (Clackson et al., Nature, 352:624-628 (1991); Felici et al., J Mol. Biol., 222:301-310 (1991); Markland et al., Gene, 109-13-19 (1991)) of the M13 bacteriophage. These antibody variable region sequences are expressed either as Fab or single chain Fv (scFv) structures at the tip of the phage carrying the respective sequences. Through rounds of a panning process using different levels of antigen binding conditions (stringencies), phages expressing Fab or scFv structures that exhibit antigen specificity and affinity comparable to the non-CDR-humanized parent antibodies (also expressed as control phage carrying Fab or scFv structures) can be selected and isolated. The antibody variable region cDNA sequences of selected phages can then be elucidated using standard sequencing procedures. These sequences will be used for the reconstruction of a full antibody with the desired isotype using established antibody engineering techniques.

2. Combining the Selected Human CDRs into One Single Antibody:

The present invention does not necessarily guarantee the identification of human sequences that can replace all six CDRs but allows for free assortments of human CDRs from a huge database into constructing a less immunogenic immunoglobulin. Even success only in replacing one murine CDR with that of a human will translate into a better and less immunogenic immunoglobulin. By doing sequential replacement of CDRs, the set of human CDR combinations that can be included into a single immunoglobulin structure will be identified and used to construct the final sequence using standard technique in molecular engineering. The variable region sequence containing human CDRs can be taken in a murine, chimeric, humanized (CDR-grafted), veneered (e.g. see U.S. Pat. No. 6,797,492, which is incorporated herein by reference), deimmunized, or framework-patched form, either as whole IgG, F(ab′)2, Fab′, Fab, monovalent sFv, or multivalent sFv, bispecific antibody, or multi-specific antibody, or as other fusion proteins etc. Because the human CDR sequence selected for humanizing the murine antibody presumably contains linear peptide screened by the human immune system as self, the resultant antibody, even by containing only one human CDR replacement, should be a better and less immunogenic antibody than its non-CDR-humanized parent.

In the event that replacing any or all of the murine immunoglobulin CDRs results in the loss of affinity and/or specificity, random mutations (including amino acid addition and deletion) at the CDR3 region of either the heavy and light chain immunoglobulin, or both, can be introduced such that partial or full recovery of the original affinity and/or specificity can be restored. Alternatively, random mutations at the CDR3 can be employed as the means for enhancing immunoreactivities above the levels of the parent immunoglobulins after CDR-humanization. The process of selecting the best CDR-humanized immunoglobulin containing mutated CDR3 can be facilitated by phage-display library and rounds of panning, as described above. Similar procedures, such as screening a ribosome-display library, can also be employed.

### 3. Expression of the CDR-Humanized Immunoglobulin:

Variable region sequences containing humanized CDRs will be genetically joined to their respective constant region sequences. Different mammalian or prokaryotic cell expression vectors can be used to express the CDR-humanized immunoglobulin using standard techniques. The immunoglobulins, including binding fragments and other derivatives thereof, of the present invention may be produced readily by a variety of recombinant DNA techniques, with ultimate expression in transfected cells, preferably immortalized eukaryotic cells, such as myeloma or hybridoma cells. The nucleic acid sequences of the present invention capable of ultimately expressing the desired CDR-humanized antibodies can be formed from a variety of different polynucleotides (genomic or cDNA, RNA, synthetic oligonucleotides, etc.) and components (e.g., V, J, D, and C regions), as well as by a variety of different techniques. Joining appropriate synthetic and genomic sequences is presently the most common method of production, but cDNA sequences may also be utilized (see, European Patent Publication No. 0239400 and Reichmann et al., Nature, 332, 323-327 (1988), both of which are incorporated herein by reference).

As stated previously, the DNA sequences will be expressed in hosts after the sequences have been operably linked to (i.e., positioned to ensure the functioning of) an expression control sequence. These expression vectors are typically replicable in the host organisms either as episomes or as an integral part of the host chromosomal DNA. Commonly, expression vectors will contain selection markers, e.g., tetracycline or neomycin, to permit detection of those cells transformed with the desired DNA sequences (see, e.g., U.S. Pat. No. 4,704,362, which is incorporated herein by reference).

E. coli is one prokaryotic host useful particularly for cloning the DNA sequences of the present invention. Other microbial hosts suitable for use include, but are not limited to, bacilli, such as Bacillus subtilus, and other enterbacteriaceae, such as Salmonella, Serratia, and various Pseudomonas species. In these prokaryotic hosts, one can also make expression vectors, which will typically contain expression control sequences compatible with the host cell (e.g., an origin of replication). In addition, any number of a variety of well-known promoters will be present, such as the lactose promoter system, a tryptophan (trp) promoter system, a beta-lactamase promoter system, or a promoter system from phage lambda. The promoters will typically control expression, optionally with an operator sequence, and have ribosome binding site sequences and the like, for initiating and completing transcription and translation. Other microbes, such as yeast, may also be used for expression. Saccharomyces is a preferred host, with suitable vectors having expression control sequences, such as promoters, including 3-phosphoglycerate kinase or other glycolytic enzymes, and an origin of replication, termination sequences and the like as desired.

In addition to microorganisms, mammalian tissue cell culture may also be used to express and produce the polypeptides of the present invention (see, Winnacker, “From Genes to Clones,” VCH Publishers, N.Y., N.Y. (1987), which is incorporated herein by reference). Eukaryotic cells are actually preferred, because a number of suitable host cell lines capable of secreting intact immunoglobulins have been developed in the art, and include the CHO cell lines, various COS cell lines, HeLa cells, preferably myeloma cell lines, etc, and transformed B-cells or hybridomas. Expression vectors for these cells can include expression control sequences, such as an origin of replication, a promoter, an enhancer (Queen et al., Immunol. Rev., 89:49-68 (1986), which is incorporated herein by reference), and necessary processing information sites, such as ribosome binding sites, RNA splice sites, polyadenylation sites, and transcriptional terminator sequences. Preferred expression control sequences are promoters derived from immunoglobulin genes, SV40, adenovirus, cytomegalovirus, bovine papilloma virus, and the like.

The vectors containing the DNA segments of interest (e.g., the heavy and light chain encoding sequences and expression control sequences) can be transferred into the host cell by well-known methods, which vary depending on the type of cellular host. For example, calcium chloride transfection is commonly utilized for prokaryotic cells, whereas calcium phosphate treatment or electroporation may be used for other cellular hosts. (See, generally, Sambrook and Russell, Molecular Cloning: A Laboratory Manual, Cold Spring Harbor Laboratory Press, (2001), which is incorporated herein by reference.)

Once expressed, the whole antibodies, their dimmers, individual light and heavy chains, or other immunoglobulin forms of the present invention, can be purified according to standard procedures of the art, including ammonium sulfate precipitation, affinity columns, column chromatography, gel electrophoresis and the like (see, generally, R. Scopes, “Protein Purification: Principles and Practice”, Springer-Verlag, N.Y. (2002)). Substantially pure immunoglobulins of at least 90 to 95% homogeneity are preferred, and 98 to 99% or more homogeneity most preferred, for pharmaceutical uses.

Hereinafter, the present invention is described in more detail by reference to exemplary embodiments. In particular, the anti-CD22 antibody RFB4 and anti-CD20 antibody 1F5 are used as examples to illustrate how a CDR can be humanized using the present invention. However, there are variations in the combinations on humanizing different heavy and light chain CDRs, and thus the following examples below are offered only to illustrate aspects of the invention and are in no way intended to limit the scope of the present invention. As such, embodiments similar or equivalent to those described herein can be used in the practice or testing of the present invention.

### Example 1

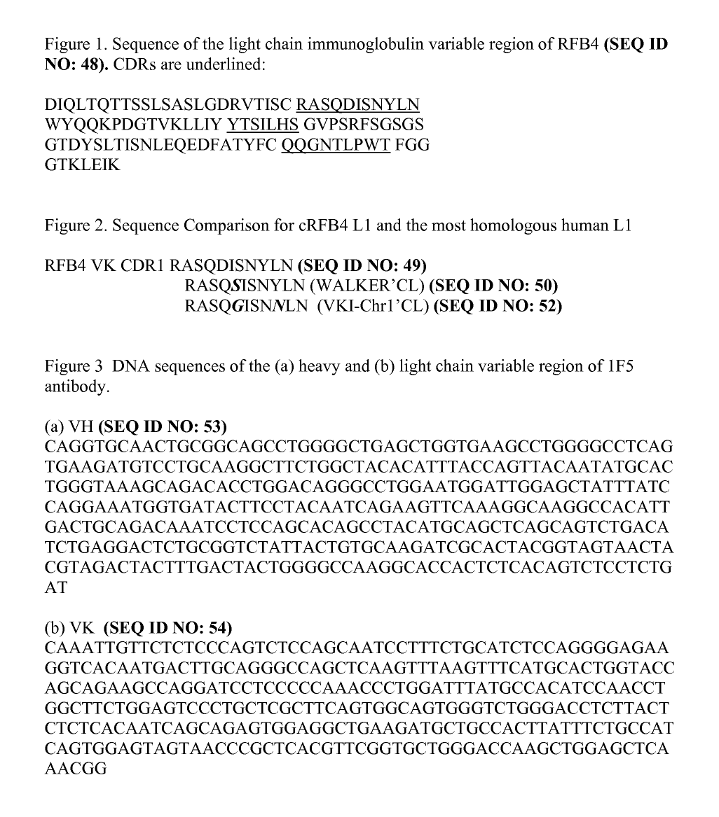

RFB4 is a publicly known antibody that targets the human CD22 antigen. The variable region sequences of the heavy (VH) and light (VL) chains are published (Mansfield et al. 1997. Recombinant RFB4 Immunotoxins Exhibit Potent Cytotoxic Activity for CD22-Bearing Cells and Tumors. Blood 80:2020-2028). The humanization of the CDR1 of RFB4 VK region in a chimeric RFB4 (cRFB4) will be used to illustrate the concept of the present invention (Yang et al. 2006. Construction and characterization of recombinant anti-B lymphoma chimeric antibody. Chinese J. New Drugs 15(3):186-192). The VK amino acid sequence is as shown in FIG. 1. The CDR1 sequence of cRFB4 VK is used to compare with the human L1 sequences in the Kabat's data base (Kabat et al. 1991. Sequences of Proteins of Immunological Interest. US Department of Health and Human Services). Two human L1 sequences with high homology to cRFB4 L1 are identified; they are from WALKER′CL and VKI-Chr1′CL (FIG. 2). The L1 sequences of WALKER′CL and VKI-Chr1′CL VK are over 90% and 80% homologous to that of the cRFB4, respectively. In the L1 sequence of human WALKER′CL, except for the D (negative charge) to S (neutral) conversion at position 28 (Numbering according to Kabat's database), at least one charged residue (R at position 24) and one aromatic residue (Y at position 32) are conserved, fulfilling the basic requirement for the selection of a human CDR. For VKI-Chr1′CL, although the L1 sequence is 80% homologous, it appears less favorable, for it carries a Y (aromatic) to N (neutral) conversion at position 32, in addition to the D→S conversion at position 28. Since these stretches of CDR1 sequences are of human origin, they presumably have been screened by the human immune system to be of non-immunogenic nature for MHC presentation.

1. Primer Design to Prepare Humanized L1 of cRFB4

PCR primers are synthesized by oligonucleotide synthesis (Molecular Informatirx Laboratory). DNA sequences encoding cRFB4 VK with its L1 replaced by that of WALKER′CL, and VKI-Chr1′CL, respectively, are prepared by overlapping polymerase chain reactions. cRFB4 VK carrying the L1 sequence of WALKER′CL is designated as 03CDR-S, and cRFB4 VK carrying the L1 sequence of VKI-Chr1′CL is designated as 03CDR-GN. The oligonucleotide primers for the PCR are listed below:

Primer 1

(SEQ ID NO: 1)

(5′-GAACTCTAGACACAGGACCTCACC-3′)
Primer S1

(SEQ ID NO: 2)

(5′-GTTCAGATAATTGCTAATGCTCTGACTTGC-3′)
Primer S2

(SEQ ID NO: 3)

(5′-AGCATTAGCAATTATCTGAACTGGTATC-3′)
and
Primer 2

(SEQ ID NO: 4)

(5′-TGCGGGATCCAACTGAGGAAG-3′)
Primer GN1

(SEQ ID NO: 5)

(5′-GTTCAGATTATTGCTAATACCCTGACTTGC-3)
Primer GN2

(SEQ ID NO: 6)

(5′-GGTATTAGCAATAATCTGAACTGGTATC-3)

### Construction of 03CDR-S:

The cRFB4 VK variable region sequence carrying a humanized L1 of WALKER′CL is assembled in halves. The N-terminal half of the VK sequence is designated as N-03CDR-S, and the C-terminal half of the VK sequence is designated as C-03CDR-S.

N-03CDR-S is PCR-amplified using Primer 1 and Primer S1, whereas C-03CDR-S is PCR-amplified using Primer S2 and Primer 2. Briefly, in a reaction volume of 50 μl containing 1× PCR buffer (Invitrogen, Carlsbad, Calif.), 1.5 mM MgCl₂ (Invitrogen), 0.2 mM dNTP (Promega, Madison, Wis.), 0.04 U/μl of Platinum Taq polymerase (Invitrogen), 50 ng of cRFB4 VK template DNA, and 0.2 μM of Primer 1 and Primer S1 (for N-03CDR-S), or Primer S2 and Primer 2 (for C-03CDR-S), PCR is performed with 3-min pre-denaturing step at 94° C., 25 extended cycles (denaturation at 94° C. for 30 s, annealing at 53° C. for 30 s, and extension at 72° C. for 30 s), followed by a 10-min post-extension step at 72° C., using a Mastercycler® personal PCR thermocycler with 25-well aluminum plate (Eppendorf, Westbury, N.Y.).

PCR products of N-03CDR-S and C-03CDR-S are joined into the L1-humanized VK sequence 03CDR-S by overlapping PCR. Briefly, 0.5 μl of the PCR products of N-03CDR-S and C-03CDR-S are mixed with 0.2 μM of Primer 1 and Primer 2. PCR reaction is carried out in a 50 μl reaction volume using similar conditions as above. The mixture is pre-denatured at 94° C. for 3 min, followed by 25 extension cycles with a Mastercycler® personal PCR thermocycler with 25-well aluminum plate (Eppendorf). Each cycle consists of denaturation at 94° C. for 45 s, annealing at 53° C. for 45 s, and extension steps at 72° C. for 45 s. After an extended incubation at 72° C. for 10 min, the PCR products are stored at 4° C. until use.

### Construction of 03CDR-GN:

The VK variable region sequence carrying a humanized L1 of VKI-Chr1′CL VK is assembled in halves. The N-terminal half of the VK sequence is designated as N-03CDR-GN, and the C-terminal half of the VK sequence is designated as C-03CDR-GN.

N-03CDR-GN is PCR-amplified using Primer 1 and Primer GN1, whereas C-03CDR-GN is PCR-amplified using Primer GN2 and Primer 2. Briefly, in a reaction volume of 50 μl containing 1× PCR buffer (Invitrogen), 1.5 mM MgCl₂ (Invitrogen), 0.2 mM dNTP (Promega), 0.04 U/μl of Platinum Taq polymerase (Invitrogen), 50 ng of cRFB4 VK template DNA, and 0.2 μM of Primer 1 and Primer QN1 (for N-03CDR-GN), or Primer QN2 and Primer 2 (for C-03CDR-GN), PCR is performed with 3-min pre-denaturing step at 94° C., 25 extended cycles (denaturation at 94° C. for 30 s, annealing at 53° C. for 30 s, and extension at 72° C. for 30 s), followed by a 10-min post-extension step at 72° C., using a Mastercycler® personal PCR thermocycler with 25-well aluminum plate (Eppendorf).

PCR products of N-03CDR-GN and C-03CDR-GN are joined into the L1-humanized sequence 03CDR-GN by overlapping PCR. Briefly, 0.5 μl of the PCR products of N-03CDR-GN and C-03CDR-GN are mixed with 0.2 μM of Primer 1 and Primer 2. PCR reaction is carried out in a 50 μl reaction volume using similar conditions as above. The mixture is pre-denatured at 94° C. for 3 min, followed by 25 extension cycles with a Mastercycler® personal PCR thermocycler with 25-well aluminum plate (Eppendorf). Each cycle consists of denaturation at 94° C. for 45 s, annealing at 53° C. for 45 s, and extension steps at 72° C. for 45 s. After an extended incubation at 72° C. for 10 min, the PCR products are stored at 4° C. until use.

2. Expression of CDR-Humanized cRFB4 Antibody

CDR-humanized VK products from overlapping PCR are purified by QIAquick PCR Purification Kit (Qiagen). The purified PCR products are subcloned into the corresponding restriction sites of the light chain expression vector, pEkappa using standard techniques in molecular biology (Yang et al. 2006. Construction and characterization of recombinant anti-B lymphoma chimeric antibody. Chinese J. New Drugs 15(3):186-192). The light chain expression vector carrying different versions of CDR-humanized cRFB4 VK will be co-transfected with the heavy chain expression vector carrying the cRFB4 VH sequence in pEgamma into SP2/0 murine myeloma cells by electroporation (Yang et al. 2006. Construction and characterization of recombinant anti-B lymphoma chimeric antibody. Chinese J. New Drugs 15(3):186-192). pEkappa carries a selection marker (hygromycin) and transfected cell can be selected for hygromycin expression. Clones secreting a maximal amount of complete antibody are detected by ELISA. Purified antibody is used to test for binding to CD22 antigen.

3. Comparison of Binding Affinity of the CDR-Humanized cRFB4 Antibody

The affinity of the CDR-humanized cRFB4 is evaluated by flow cytometry. Raji cells (5×10⁵) are incubated with 1 μg of either purified cRFB4 or cRFB4 carrying humanized CDRs in a final volume of 100 μl of PBS supplemented with 1% FCS and 0.01% (w/v) sodium azide (PBS-FA). The mixtures are incubated for 30 minutes at 4° C. and washed three times with PBS to remove unbound antibodies. The binding levels of the antibodies to Raji cells are assessed by the addition of a 20× diluted FITC-labeled, goat anti-human IgG1, Fc fragment-specific antibodies (Jackson ImmunoResearch, West Grove, Pa.) in a final volume of 100 μl in PBS-FA, and incubating for 30 minutes at 4° C. The mixture is washed three times with PBS and fluorescence intensities are measured by a FACSCAN fluorescence-activated cell sorter (Becton Dickinson, Bedford, Mass.).

To further compare the affinity of the antibody before and after CDR-humanization, a competitive binding assay is performed. Fixed amount (10× dilution from stock) of FITC-conjugated RFB4 (Ancell Corporation, Bayport, Minn.) is mixed with varying concentrations of either cRFB4 or CDR-humanized cRFB4. The mixtures are added to Raji cells in a final volume of 100 μl in PBS-FA, and incubated for 30 minutes at 4° C. After washing three times with PBS, the fluorescence intensities of Raji cells bound with the FITC-RFB4 are measured by FACSCAN (Becton Dickinson, Bedford, Mass.).

### Example 2

1F5 is another publicly known antibody that targets the human CD20 antigen. The variable region sequences of the heavy (VH) and light (VL) chains are published (Shan et al. 1999. Characterization of scFv-Ig constructs generated from the anti-CD20 mAb 1F5 using linker peptides of varying lengths. J. Immuol. 162:6589-6595). The variable region sequences of 1F5 is obtained either by oligonucleotide-based gene synthesis techniques with the published sequences, or alternatively directly from the 1F5 hybridoma as follows:

1. Briefly, the hybridoma for 1F5 was obtained from American Type Culture Collection (ATCC#HB-9645; lot #221900). RNA was extracted from 3×10⁷ hybridoma cells using a Track mRNA Isolation Kit (Invitrogen). cDNA was then prepared by a cDNA Cycle Kit (Invitrogen) using the primer CH1B (5′ ACA GTC ACT GAG CTG G 3′) (SEQ ID NO: 7) that is specifically prime to the CH1 constant region of mouse IgG and the primer Ck3BH1 (5′ GCC GGA TCC TCA CTG GAT GGT GGG AAG ATG GAT ACA 3′) (SEQ ID NO: 8) that is prime to the kappa constant region of mouse IgG. The first-strand cDNA were then amplified by RACE and PCR methods. The DNA fragments after PCR were cloned into a TA Cloning Vector (Invitrogen) and then sequenced by the dideoxy chain termination method. The DNA and amino acid sequences of 1F5 heavy and light chain variable genes are shown in FIGS. 3 and 4, respectively.

2. Functional Humanization of 1F5 by Framework-Re-Engineering:

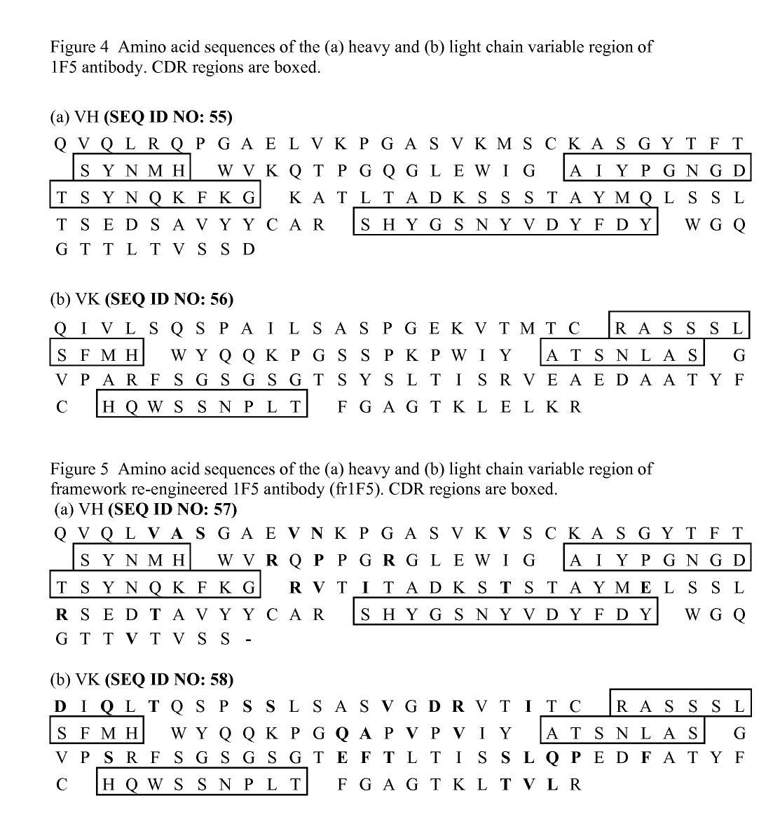

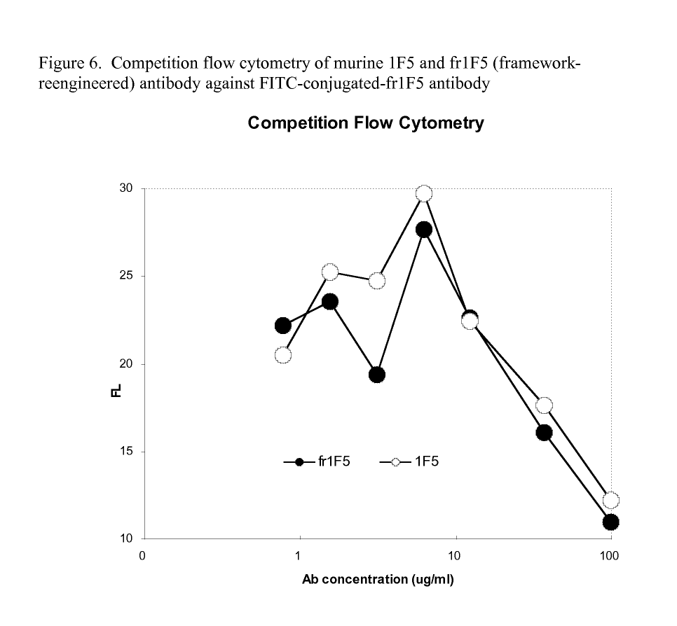

The 1F5 antibody was functionally humanized using a technique known as framework-re-engineering, in which, framework segments (FR1, FR2, FR3, FR4) from different human immunoglobulins were freely assorted to obtain the best-fit scaffold supporting the murine CDRs. The design and construction of the framework-re-engineered 1F5 were as described in Lei et al (U.S. Provisional Patent Application No. 60/878,030 and International Application No. PCT/IB07/004379, the contents of which are hereby incorporated by reference). The VH sequence of the framework-re-engineered 1F5 is composed of LS2′CL (FR1)-CDR1-NEWM (FR2)-CDR2-783C′CL (FR3)-CDR3-4G12′CL (FR4) (FIG. 5A); and the VK sequence of the framework-re-engineered 1F5 is composed of BJ19 (FR1)-CDR1-MOT (FR2)-CDR2-WES (FR3)-CDR3-NIG-58 (FR4) (FIG. 5B). The designed VH and VK sequence for the framework re-engineered 1F5 were assembled by a combination of oligonucleotide synthesis and PCR. The framework-re-engineered 1F5 antibody was demonstrated to exhibit immunoreactivity comparable to that of its murine counterpart (FIG. 6).

3. Although the 1F5 antibody was successfully re-engineered using the approach of functional humanization, the CDRs are still of murine origin and may be potential sources of immunogenicity. Reduction of immunogenicity attributed by the murine CDRs using the present invention is done as follows:
a. the heavy chain CDR 1 of 1F5 has the shortest sequence (SYNMH) (SEQ ID NO: 9), and will be the first CDR to be humanized;
b. the light chain CDR2 of 1F5 has the second shortest sequence (ATSNLAS) (SEQ ID NO: 10), and will be the second CDR to be humanized.

Humanization of H1 and L2 will be used as examples for illustration. Humanization of other CDRs of the heavy and light chain can be extended using similar principles.

4. Humanization of the H1 of 1F5:

The DNA sequence encoding murine H1 of 1F5 was entered into Blast-search using the NCBI IgBlast database. A total of 9 human Ig sequences of high nucleotide homology were identified. These highly homologous nucleotide sequences were translated into amino acid sequences using the Proteomics and sequence analysis tools (cn.expasy.org/tools) from Expert Protein Analysis System (ExPASy) homepage. Sequence alignment of all translated sequences was performed by cluster analysis using the ClustalW software from Pôle BioInformatique Lyonnais (pbil.univ-lyonl.fr/). The results of the alignment are as shown below (SEQ ID NOS 11-20):

| | |
|---|---|
| AF376954 | NYNMH |
| AC110080 | KYNMH |
| AY429737 | GYNMH |
| AJ407992 | GYNMH |
| AF087418 | GSNMH |
| Test 1 × 0 | SYNHM |
| AF376951 | SYNMH |
| DQ926652 | SYYMH |
| AC148025 | SYNLH |
| AP001241 | SFNMQ |
| | :: |
| Prim. cons. | SYNMH |

Multiple Alignments of Homologous Human H1 with that of 1F5

As can be seen from the aligned sequences, the sequence of the H1 of 1F5 is virtually identical to that of human H1 of AF376951 (Homo Sapiens, Clone MEI Immunoglobulin heavy chain variable region). Based on the principles and rationales of the present invention, the heavy chain H1 of 1F5 is by default a human H1. There is no need to make further modifications within the H1 sequence; or alternatively, the H1 of 1F5 was humanized without the need to make further modifications.

Nevertheless, for the purpose of illustration using the principles and selection criteria as set forth in the present invention, two other human H1 sequences obtained from the Blast search, in addition to the original H1 sequence, are used for humanizing the H1 of the framework re-engineered 1F5 antibody.

| | |
|---|---|
| 31 32 33 34 35 | |
| S Y N M H (AF376951) | (SEQ ID NO: 17) |
| S Y [symbol] M H (DQ926652) | (SEQ ID NO: 18) |
| S Y N [symbol] H (AC148025) | (SEQ ID NO: 19) |
| S Y N M H (1F5) | (SEQ ID NO: 21) |

The H1 sequence of 1F5 carries an aromatic tyrosine (Y) and a basic histidine (H). In the selection of appropriate human H1 for 1F5 H1-humanization, all of the human H1 sequences contain Y and H at corresponding positions. Since the H1 sequence of AF376951 is identical to that of 1F5, its selection for H1-humanization is an obvious choice. An analysis of the heavy chain CDR sequences shows that the residue at position 31 (Kabat's numbering: that is the first residue of H1) was directly involved in ligand binding in 13 out of 31 complexes. The residue at position 32 was found to interact with ligand in eight cases; in three of these, only main chain atoms were involved. The residue at position 34 was not directly involved in ligand contact in any of the 31 complexes. Therefore, in the H1 of 1F5, residue 31 (S) is likely to be a residue important for maintaining the binding site structure/contacts of the immunoglobulin, and, residue at position 34 (M) is likely to be inconsequential to ligand binding. Therefore, in the choice of the human H1 for CDR-humanization, efforts are made to maintain the residue at position 31, while modification of the residue at position 34 is loosely allowed.

With this in mind, for the H1 sequence of DQ926652, asparagine (N), the amide derivatives of acidic amino acid, is replaced by an aromatic Y; this can cause significant changes in the contacts and conformation of the resultant antigen binding site, it . Yet, based on the degree of homology (80%) and the fact that the original Y at position 32, H at position 35 and the important S (determined by computer database analysis based on X-ray crystallographic information) at position 31 are retained, it is considered as a possible choice. For the H1 sequence of AC148025, the S in position 31 suggested to be important by database analysis is retained. Although the sulfur-containing methionine (M) is replaced by the aliphatic leucine (L), which is of similar size to M, the position (34) was determined to be inconsequential to ligand binding in most cases. Such conversion is mild, and based on the criteria of sequence homology (80%) and conservation of aromatic and charged structures, the H1 sequence is chosen as the candidate for humanizing the H1 of 1F5.

5. Humanization of the L2 of 1F5:

The DNA sequence encoding the murine L2 of 1F5 was entered into Blast-search using the NCBI IgBlast database. A total of 5 human Ig sequences of high nucleotide homology were identified. These highly homologous nucleotide sequences were translated into amino acid sequences using the Proteomics and sequence analysis tools (cn.expasy.org/tools) from Expert Protein Analysis System (ExPASy) homepage. Sequence alignment of all translated sequences was performed by cluster analysis using the ClustalW software from Pôle BioInformatique Lyonnais (pbil.univ-lyonl.fr/). The results of the alignment are as shown below (SEQ ID NOS 22-27):

| | |
|---|---|
| AC034151 | TTSSLAR |
| AC128677 | TTSSLAR |
| AC002060 | SKSILAS |
| ACO16745 | TTSNMAD |
| Test 2 × 0 | ATSNLAS |
| AC103563 | ATPNLDC |
| | :.. : |
| Prim. cons. | TTSNLA2 |

Multiple Alignments of Homologous Human L2 with that of 1F5

Based on the above sequence alignment, and using the criteria as described previously, only two of the sequences were selected from this blast search for L2 humanization. They are: SKSILAS (SEQ ID NO: 24) (Homo sapiens Chromosome 22q 11.2 BAC Clone 142e2 In IGLC Region, complete sequence; accession number: AC002060) and TTSNMAD (SEQ ID NO: 25) (Homo sapiens BAC clone RP11-480C16 from 2, complete sequence; accession number: AC016745).

A search in the Kabat's database (Kabat et al. 1991. Sequences of proteins of immunological interest. 5^{th} Edition. US Dpt of Health.) had revealed another human light chain sequence that could be used for CDR-humanization. It is: AASNLQS (SEQ ID NO: 28) from GAL(I) of human kappa light chain subgroup I.

| | |
|---|---|
| 50 51 52 53 54 55 56 | |
| [symbol] T S N [symbol] A [symbol] (AC016745) | (SEQ ID |
| | NO: 25) |
| [symbol] [symbol] S [symbol] L A S (AC002060) | (SEQ ID |
| | NO: 24) |
| A [symbol] S N L [symbol] S (GAL(I)) | (SEQ ID |
| | NO: 28) |
| A T S N L A S (1F5) | (SEQ ID |
| | NO: 26) |

The L2 sequence of 1F5 does not carry aromatic or charged (basic or acidic) structures. Sequence AC016745 is about 57% homologous to the 1F5 VK CDR2 sequence. In the L2 sequence of AC016745, the A to T conversion at position 50 (Kabat's numbering), and L to M conversion at position 54 are somewhat conservative, except that T contains a hydroxyl group and M is sulphur containing. The S to D conversion at position 56 perhaps is the most drastic change as D is negatively charged at physiological conditions. Therefore, L2 sequence from AC016745 may not be the most favorable sequence for humanizing the L2 of 1F5. The L2 sequence in AC002060 is about 71% homologous to that of 1F5. The T (hydroxyl group-containing) to K (basic) conversion at position 51 and N (amid derivative) to I (aliphatic) conversion at position 53 are significant changes, and may affect the immunoreactivity of the resultant immunoglobulin. The L2 sequence of GAL (I) is about 71% homologous to that of 1F5. The T (hydroxyl group-containing) to A (aliphatic) conversion at position 51 is considered mild, yet the A (aliphatic) to Q (amide derivative) conversion at position 55 is more drastic. Since these conversions appear at different positions along the L2 sequence, their impacts on the final immunoreactivity of the CDR-humanized immunoglobulin will not be known without experimentation. Since these stretches of L2 are of human origin, again, they presumably should have been screened and tested by the human immune system to be of non-immunogenic nature for MHC presentation.

6. Construction of the Various CDR-Humanized VH and VK Sequences for Framework-Re-Engineered 1F5:

VH: For the H1-humanization of the framework-re-engineered1F5, the three most homologous human H1 chosen would be that of AF376951 (Homo Sapiens, Clone MEI Immunoglobulin heavy chain variable region) (SYNMH) (SEQ ID NO: 9), DQ926652 (SYYMH) (SEQ ID NO: 18), and AC148025 (SYNLH) (SEQ ID NO: 19).

The original H1 of 1F5 is identical in sequence to AF376951, and is by default a humanized H1 without the need for further modification.

In order to examine the general applicability of modifying H1 sequence, the two other most homologous human H1 that fulfill the selection criteria above were also used to construct H1-humanized immunoglobulin.
V1 version was CDR-humanized with the human H1 from DQ926652
V2 version was CDR-humanized with the human H1 from AC148025

Their constructions were done with the following primers:

5′NH-LG:

(SEQ ID NO: 29)

GTG CAA CTG CAG GCT TCC GGG GCT GAG GTA AAT AAG
CCT GGG GCC TCA GTG AAG
3′NH-LG-v1:

(SEQ ID NO: 30)

TAC CCA GTG CAT ATA GTA ACT GGT AAA TGT
3′NH-LG-v2

(SEQ ID NO: 31)

TAC CCA GTG CAA ATT GTA ACT GGT AAA TGT
5′CH-LG-v1

(SEQ ID NO: 32)

AGT TAC TAT ATG CAC TGG GTA CGG CAG CCT
5′CH-LG-v2

(SEQ ID NO: 33)

AGT TAC AAT TTG CAC TGG GTA CGG CAG CCT
3′CH-LG

(SEQ ID NO: 34)

GGA GAC GGT GAC CGT GGT GCC TTG GCC CCA GTA GTC
AAA GTA GTC TAC GT

The V1 version was constructed in halves and connected by overlapping PCR using the primers 5′NH-LG, 3′NH-LG-v1, 5′CH-LG-v1 and 3′CH-LG. Briefly, in a reaction volume of 50 μl containing 1× PCR buffer (Invitrogen), 1.5 mM MgCl₂ (Invitrogen), 0.2 mM dNTP (Promega), 0.04 U/μl of Platinum Taq polymerase (Invitrogen), 50 ng of framework-re-engineered 1F5 VH template DNA, and 0.2 μM of 5′NH-LG and 3′NH-LG-v1(N-terminal half), or 5′CH-LG-v1 and 3′CH-LG (C-terminal half), PCR was performed with 3-min pre-denaturing step at 94° C., 25 extended cycles (denaturation at 94° C. for 30 s, annealing at 53° C. for 30 s, and extension at 72° C. for 30 s), followed by a 10-min post-extension step at 72° C., using a Mastercycler® personal PCR thermocycler with 25-well aluminum plate (Eppendorf).

PCR products of N-terminal half and C-terminal half of version 1 were joined into the humanized VH sequence by overlapping PCR. Briefly, 0.5 μl of the PCR products of N-terminal half and C-terminal half of version 1 were mixed with 0.2 μM of 5′NH-LG and 3′CH-LG. PCR reaction was carried out in a 50 μl reaction volume using similar conditions as above. The mixture was pre-denatured at 94° C. for 3 min, followed by 25 extension cycles with a Mastercycler® personal PCR thermocycler with 25-well aluminum plate (Eppendorf). Each cycle included the steps of denaturation at 94° C. for 45 s, annealing at 53° C. for 45 s, and extension steps at 72° C. for 45 s. After an extended incubation at 72° C. for 10 min, the PCR products were stored at 4° C. until use.

Construction of V2 was basically identical to the construction of V1 except that all steps involving the oligonucleotide primers of 5′CH-LG-v1 and 3′NH-LG-v1 were done by replacing these oligonucleotide primers with 5′CH-LG-v2 and 2′NH-LG-v2, respectively.
VK: For the L2-humanization of the framework-re-engineered1F5, the three most homologous human L2 chosen would be that of AC002060 (SKSILAS) (SEQ ID NO: 24), AC016745 (TTSNMAD) (SEQ ID NO: 25), and GAL(I) (AASNLQS) (SEQ ID NO: 28).
V3 version was CDR-humanized with the human L2 from AC002060
V4 version was CDR-humanized with the human L2 from AC016745
V5 version was CDR-humanized with the human L2 from GAL(I)

Their constructions were done with the following primers:

5′NK-LG:

(SEQ ID NO: 35)

GAT ATT CAG CTG ACA CAG TCT CCA TCA AGT CTT TCT
GCA TCT GTG
3′NK-LG-v3:

(SEQ ID NO: 36)

GGA AGC CAG GAT GGA CTT GGA ATA AAT TAC
3′NK-LG-v4

(SEQ ID NO: 37)

ATC AGC CAT GTT GGA TGT GGT ATA AAT TAC
3′NK-LG-v5

(SEQ ID NO: 38)

GGA CTG CAG GTT GGA GGC GGC ATA AAT TAC
5′CK-LG-v3

(SEQ ID NO: 39)

TAT TCC AAG TCC ATC CTG GCT TCC GGA GTC CCT
5′CK-LG-v4

(SEQ ID NO: 40)

ACC ACA TCC AAC ATG GCT GAT GGA GTC CCT
5′CK-LG-v5

(SEQ ID NO: 41)

GCC GCC TCC AAC CTG CAG TCC GGA GTC CCT
3′CK-LG

(SEQ ID NO: 42)

CCG TTT GAT CAC CAG CTT GGT CCC AGC ACC GAA CGT
GAG CGG

The V3 version was constructed in halves and connected by overlapping PCR using the primers 5′NK-LG, 3′NK-LG-v3, 5′CK-LG-v3 and 3′CK-LG. Briefly, in a reaction volume of 50 μl containing 1× PCR buffer (Invitrogen), 1.5 mM MgCl₂ (Invitrogen), 0.2 mM dNTP (Promega), 0.04 U/μl of Platinum Taq polymerase (lnvitrogen), 50 ng of framework-re-engineered 1F5 VK template DNA, and 0.2 μM of 5′NK-LG and 3′NK-LG-v3(N-terminal half), or 5′CK-LG-v3 and 3′CK-LG (C-terminal half), PCR was performed with 3-min pre-denaturing step at 94° C., 25 extended cycles (denaturation at 94° C. for 30 s, annealing at 53° C. for 30 s, and extension at 72° C. for 30 s), followed by a 10-min post-extension step at 72° C., using a Mastercycler® personal PCR thermocycler with 25-well aluminum plate (Eppendorf).

PCR products of N-terminal half and C-terminal half of version 3 were joined into the humanized VK sequence by overlapping PCR. Briefly, 0.5 μl of the PCR products of N-terminal half and C-terminal half of version 3 were mixed with 0.2 μM of 5′NK-LG and 3′CK-LG. PCR reaction was carried out in a 50 μl reaction volume using similar conditions as above. The mixture was pre-denatured at 94° C. for 3 min, followed by 25 extension cycles with a Mastercycler® personal PCR thermocycler with 25-well aluminum plate (Eppendorf). Each cycle consists of denaturation at 94° C. for 45 s, annealing at 53° C. for 45 s, and extension steps at 72° C. for 45 s. After an extended incubation at 72° C. for 10 min, the PCR products were stored at 4° C. until use.

Construction of V4 and V5 was basically identical to the construction of V3 except that all steps involving the oligonucleotide primers of 5′CK-LG-v3 and 3′NK-LG-v3 were done by replacing these oligonucleotide primers with 5′CK-LG-v4 and 3′NK-LG-v4 for Version 4; and 5′CK-LG-vS and 3′NK-LG-vS for Version 5, respectively.

7. Expression of the Various CDR-Humanized Anti-CD20 Antibody as scFv-Phage Antibody:

Variable immunoglobin (Ig) VH and VL sequences, that contain different versions of humanized CDR were joined together to form single chain variable fragment (scFv) of antibody by overlap PCR.

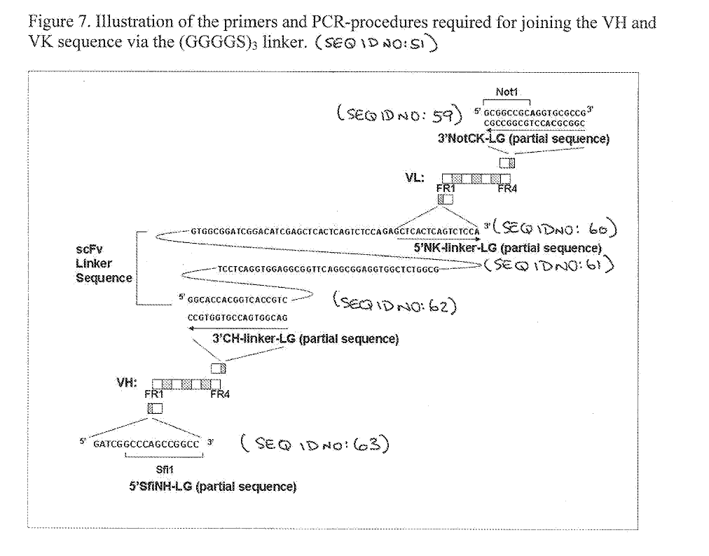

The VH and VL sequences were joined via a peptide linker with the sequence of (GGGGS)₃ (SEQ ID NO: 51) (variations in the linker sequence and size are possible). See FIG. 7.

The different versions of CDR-humanized VH and VK sequences were first PCR amplified with 5′SfiNH-LG and 3′CH-linker-LG, and 5′NK-linker-LG and 3′Not1CK-LG, respectively. Their sequences are listed below:

5′SfiNH-LG (Sfi1 restriction site underlined)

(SEQ ID NO: 43)

GAT CGG CCC AGC CGG GCC GTG CAA CTG CAG GCT TCC
3′GH-linker-LG (Partial linker sequence in italic)

(SEQ ID NO: 44)

GAC GGT GAC CGT GGT GCC GGA GAc GGT GAc cGT GGT
5′NK-linker-LG (Partial linker sequence in italic)

(SEQ ID NO: 45)

GAG CTC ACT CAG TCT CCA GAT ATT CAG CTG ACA CAG
3′Not1CK-LG (Not1 restriction site underlined)

(SEQ ID NO: 46)

CGG CGC ACC TGC GGC CGC CCG TTT GAT CAC CAG CTT

The different versions of CDR-humanized VH were PCR amplified with the primer set 5′SfiNH-LG and 3′CH-linker-LG to introduce a Sfi1 cloning site and partial linker sequence, and the different versions of CDR-humanized VK were PCR-amplified with the primer set 5′NK-linker-LG and 3′Not1CK-LG to introduce a Not1 cloning site and partial linker sequence. PCR was carried out in 50 μl of reaction volume containing 1× PCR buffer (Invitrogen); 1.5 mM MgCl₂ (Invitrogen); 0.2 mM dNTP (Promega); 0.04 U/μl of Platinum Taq polymerase (Invitrogen); 50 ng of PCR products of different versions of CDR-humanized V_{H} or V_{κ}, respectively, and 0.2 μM of the corresponding primers. After 3-min pre-denaturing step at 94° C., 25 extension cycles were carried out in a Mastercycler® personal PCR thermocycler with 25-well aluminum plate (Eppendorf). Each cycle consisted of denaturation at 94° C. for 40 s, annealing at 50° C. for 40 s, and extension at 72° C. for 40 s, followed by a 10-min post-extension step at 72° C., and 5 μl of PCR product of each VH and VL containing partial linker sequence and a unique cloning site was used as template for the overlap PCR.

The overlap PCR was carried out in 50 μl of reaction mixture containing 1× PCR buffer (lnvitrogen), 2.5 mM MgCl₂ (Invitrogen), 0.2 mM dNTP (lnvitrogen), 0.04 U/μl of Platinum Taq polymerase (Invitrogen), 5 μl of PCR products of the different versions of CDR-humanized VH and VK, 0.2 μM of flanking primers (5′SfiNH-LG and 3′Not1CK-LG), and 0.02 μM of scFv linker oligonucleotide (GGC ACC ACG GTC ACC GTC TCC TCA GGT GGA GGC GGT TCA GGC GGA GGT GGC TCT GGC GGT GGC GGA TCG GAC ATC GAG CTC ACT CAG TCT CCA GAG CTC ACT CAG TCT CCA) (SEQ ID NO: 47) which encodes the linker sequence (GGGGS)₃ (SEQ ID NO: 51). The mixture was pre-denatured at 94° C. for 3 min, followed by 25 extension cycles with a Mastercycler® personal PCR thermocycler with 25-well aluminum plate (Eppendorf). Each cycle consisted of denaturation at 94° C. for 60 s, annealing at 50° C. for 60 s, and extension steps at 72° C. for 60 s. After an extended incubation at 72° C. for 10 min, the PCR product (scFv) was stored at 4° C. until use.

After overlap PCR, single chain variable fragments (scFv) were purified by QIAquick PCR Purification Kit (Qiagen). The purified PCR products were subjected to Sfi I digestion in 1× NEBuffer 2 (New England Biolabs) supplemented with 0.01% BSA (1× NEB-BSA, New England Biolabs), 5 U of Sfi I restriction enzyme, and 1 ug of purified scFv in a reaction volume of 50 μl. Reaction mixture was incubated at 37° C. incubator for overnight. After Sfi I digestion, the digested product was purified by QIAquick Nucleotide Removal Kit (Qiagen) and then subjected to Not I digestion in 1× NEBuffer 3 (New England Biolabs) supplemented with 0.01% BSA (1× NEB-BSA, New England Biolabs), 5 U of Not I restriction enzyme, and purified Sfi 1-digested scFv in a reaction volume of 50 μl. Reaction mixture was incubated overnight at 37° C. The Sfi I/Not I-digested scFv was gel-purified (QIAquick Gel Extraction Kit; Qiagen) for subsequent subcloning steps.

Phagemid pCANTAB 5E (Amersham) was linearized by Sfi I and Not I double digestion, for subcloning of the digested scFv sequences into the corresponding sites. Ligation was then carried out in 1× T4 ligation buffer (Invitrogen) with a vector:insert molar ratio of 1:3 and with a total DNA concentration <100 ng.

The ligated DNA constituted a scFv phage (scFv-pCANTAB 5E). In theory, the different versions of CDR-humanized VH and VK could be freely combined and mixed to form a scFv library. For illustration purpose, scFv phage containing different VH and VL combinations were individually assembled. These phages included the following combinations: VH(original):VL(original), VH:VL(version 3), VH:VL(version 4); VH:VL(version 5); VH(version 1):VL(original); VH(version 1):VL(version 3); VH(version 1):VL(version 4); VH(version 1):VL(version 5); VH(version 2):VL(version 3); VH(version 2):VL(version 4); VH(version 2):VL(version5). Each scFv-phage DNA was introduced into E. coli TG1 (Stratagene) by electroporation. Briefly, 2 μl of scFv-pCANTAB 5 E was mixed with 20 μl of TG1 electroporation-competent cells (Stratagene) and placed in a sterile electroporation cuvette (0.1-cm-gap) (BioRad). After pulsing the sample once (2000V, 25 μF, and 200Ω), 1 ml of SOC medium was added to resuspend the cells. The cells were then transferred to a sterile 14-ml BD Falcon polypropylene round-bottom tube (BD Biosciences) and incubated at 37° C. for 1 hr with shaking at 250 rpm.

The culture was centrifuged at 4,000 rpm at 4° C. for 5 min. The cell pellet was re-suspended in 10 ml of SOBG medium containing 100 μg/ml ampicillin and 5 mM MgCl₂ and then incubated on ice for 15 min with gentle occasional shaking. The number of cells was determined by spectrophotometry at 600 nm, with an estimation of OD₆₀₀ at 0.4 equals 10⁸ cells/ml.

M13KO7 helper phage (Amersham) was added to the cell suspension at a multiplicity of infection (moi) ratio of 3:1 and the infection of M13KO7 helper phage was carried out at 37° C. for 30 min without shaking and then at 37° C. for 30 min with shaking at 200 rpm. After incubation, the infected culture was centrifuged at 4,000 rpm at 4° C. for 10 min and cell pellet was re-suspended in 10 ml of 2X-YT medium containing 100 μg/ml ampicillin and 50 μg/ml kanamycin. Rescue efficiency was determined by spreading a serial dilution (1×10⁻¹×, 10⁻²×, and 10⁻³×) of cell suspension onto SOBAG-K plate (SOBG medium with 1.5% Bacto-agar, 100 μg/ml ampicillin, and 50 μg/ml kanamycin), and incubated at 37° C. overnight (>20 hr). Remaining cell suspension was made up to 50 ml with 2X-YT medium containing 100 μg/ml ampicillin and 50 μg/ml kanamycin, and incubated at 37° C. overnight with shaking at 250 rpm for recombinant scFv-phage production.

8. Comparing the Binding Affinities of scFv Phage Antibody Containing Different Combinations of VH and VL on Raji Cell Surface Antigen

scFv-phage in the culture supernatants were quantified using the following equation:

$$Number of Phage particles per ml = \frac{( {OD}_{269} - {OD}_{320} ) \times ( 6 \times {10}^{16} )}{\begin{matrix}Number of nucleotides \\ in the phage genome\end{matrix}}$$

To avoid absorbance interference at OD₂₆₉ and OD₃₂₀, phages were first purified by PEG/NaCl precipitation before absorbance measurement.

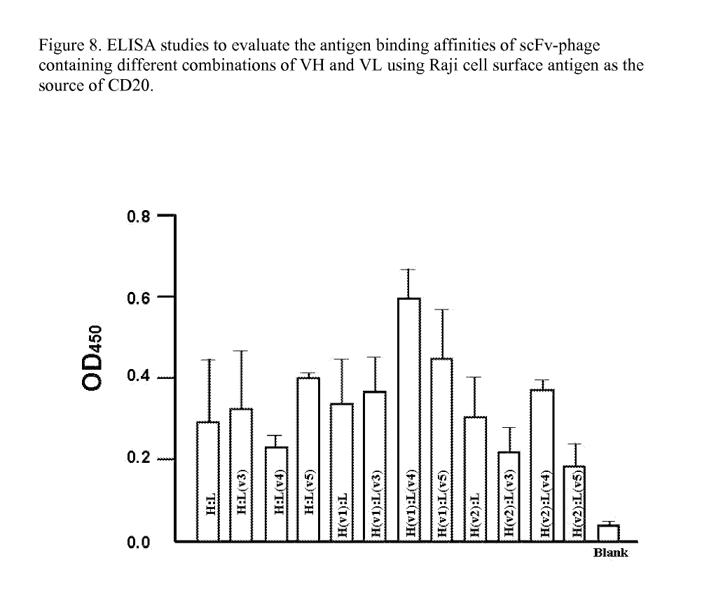

The ELISA binding studies were performed with Raji cell membrane protein as antigen. The preparation of Raji cell membrane antigen was done as follows. Briefly, 1×10⁷ Raji cells were pelleted by centrifugation, and the cell pellet was resuspended in 0.5 ml of PBS. The cell suspension was then sonicated on ice, and cell debris was removed by centrifugation. The protein concentration of the supernatant containing Raji cell membrane antigens was determined using BCA assays. To ELISA plate coated with Raji cell membrane antigens, supernatants (100 μl) containing equal amounts of different scFv-phage constructs were added, and the ELISA plate was incubated at 37° C. for 1 hour. After washing the ELISA plate three times with PBS, HRP-conjugated murine antibody specific for M13 phage (Amersham) was added to reveal the amount of antigen bound phage in the wells of the ELISA plate (FIG. 8).

9. Panning of scFv-Phage-Displayed Library for CD20 Antigen

In the event that a library of CDR-humanized anti-CD20 scFv-phage antibodies is constructed, all cultures transformed with the scFv-phage are pooled and expanded as described above. Panning procedures are introduced to identify CD20-binding scFv-phage. Briefly, the recombinant scFv-phage containing supernatant is transferred into a 50-ml ice-cold centrifuge tube, followed by the addition of 5 ml of PEG/NaCl [20% polyethylene glycol, PEG, M.W. 8000 (Sigma), and 2.5 M NaCl (Sigma)] per 25 ml of supernatant. After placing on ice for 1.5 hr, the mixture is centrifuged at 10,000×g at 4° C. for 30 min to collect the precipitated recombinant phages. Phage pellet is resuspended in 2 ml of 2X-YT medium containing 1% BSA. To determine scFv-phage titer, 2 μl of re-suspended recombinant phages is taken out and serially diluted with 200 μl of 2X-YT medium (10⁻²×, 10⁻⁴×, 10⁻⁶×, 10⁻⁸×, and 10⁻¹⁰×). From each dilution, 2 μl of diluted recombinant phage is taken out and added into 200 μl of log-phase E. coli TG1, which are then incubated at 37° C. for 30 min (without shaking) for recombinant phage infection.

Log-phase TG1 is prepared by inoculating 10 ml of 2X-TY medium containing 5 mM MgCl₂ with 100 μl of TG1 overnight culture. The inoculum is incubated at 37° C. (with shaking at 250 rpm) until OD₆₀₀ absorbance between 0.4 and 0.5. The bacterial culture is then pre-chilled on ice for 20 min before use. After recombinant phage infection, 100 μl of TG1 infected cells are spread onto SOBAG plate (SOBG medium with 1.5% Bacto-agar, and 100 μg/ml ampicillin), and incubated at 30° C. for overnight.

For the remaining concentrated recombinant phage, 2 volumes of scFv-phage blocking buffer [1× PBS, 0.2% Triton X-100 (Sigma), 0.01% NaN₃ (Riedel-de Haën), 0.1% BSA (Sigma), and 10% non-fatted milk (Nestle)] is added. After pre-blocking at room temperature for 30 min, 0.5 ml of diluted recombinant phage in blocking buffer is added into each well of 24-well culture plate (Corning) coated with the CD20 antigen (Abnova GmbH, Heidelberg, Germany. H00000931-P01/MS4A1 Recombinant Protein P01)

Pre-blocked plate is prepared one day before the experiment by dissolving CD20 antigen in carbonate coating buffer, pH 9.6, (15 mM Na₂CO₃ (Sigma) and 35 mM NaHCO₃ (Sigma)) in a final concentration of 10 μg/ml of antigen and with each well coated with 1 ml of the antigen-containing carbonate coating buffer. After overnight incubation at 4° C. with gentle shaking, each well is washed 3 times with 3 ml of borate washing buffer at pH 8.0 [26 mM Na₂B₄O₇ (BDH), 100 mM H₃BO₃ (Sigma), 0.1% BSA (Sigma), 100 mM NaCl (Sigma), 3 mM KCl (Sigma), and 0.5% Tween-20 (USB)]. After washing, each well is blocked with 2.5 ml of the same buffer at 37° C. for 2 hr, and then 3 times of washes with 3 ml of borate washing buffer before panning.

Panning is performed by incubation at room temperature for 2 h with gentle shaking. After the removal of unbound scFv-phage, the wells are washed with 1× PBS for 5 times with vigorous shaking for 30 s each time. The wells are then washed with 2.5 ml PBS containing 0.1% Tween-20 (USB) for 10 times. After washing, bound scFv-phages are eluted with 10 minute-incubation of 100 μl of 0.1 M glycine-HCl, pH 2.2. After elution, the acid is immediately neutralized with 10 μl of 1 M Tris-HCl, pH 8.0.

All the eluted scFv-phages are pooled and transferred into 50 ml of log-phase E. coli TG1 containing 2% glucose and 5 mM MgCl₂ for re-infection. Re-infection is carried out at 37° C. for 30 min without shaking and then 30 min at 37° C. with shaking at 200 rpm. The titer of panning output is determined by spreading 100 μl of re-infected TG1 culture onto SOBAG plate at a dilution of 1×, 10⁻¹×, 10⁻²×, and 10⁻³×, and then incubating at 30° C. for overnight.

The remaining re-infected culture is used and rescued with M13KO7 helper phage by adding a final concentration of 100 μg/ml ampicillin and 5×10⁹ pfu/ml M13KO7 helper phage into the re-infected culture. Super-infection is carried out for 30 min at 37° C. without shaking and then 30 min at 37° C. with shaking at 200 rpm. Rescued culture is placed on ice for 10 min and then centrifuged at 4,000 rpm at 4° C. for 10 min. The rescued cell pellet is re-suspended in 50 ml of 2X-YT medium containing 100 μg/ml ampicillin, and 50 kanamycin. The titer of next round panning input is determined by spreading 100 μl of rescued culture, in serial dilutions of 1×, 10⁻¹×, 10⁻²×, and 10⁻³×, onto SOBAG-K plate, and incubated at 37° C. overnight (>20 hr). The remaining rescued culture is incubated with shaking at 250 rpm at 37° C. overnight to produce recombinant phage for the next round of panning and the panning process is repeated twice, with a 10-fold reduction of antigen concentration coated in each round of panning.

After two rounds of panning, the screening process is completed by re-infection of the eluants of second round with 50 ml of log-phase TG1 culture containing 2% glucose and 5 mM MgCl₂. The mixture is then incubated at 37° C. without shaking and then 37° C. with shaking at 200 rpm. Panning output is determined by spreading 100 μl of re-infected culture onto SOBAG plate in 1×, 10⁻¹×, 10⁻²× and 10⁻³× dilutions. The remaining re-infected culture is recovered by centrifugation at 4,000 rpm at 4° C. for 10 min and the cell pellet is re-suspended in 8 ml of 2X-YT medium with 20% glycerol (Sigma) and then stored at −70° C. in aliquots.

The antigen specificity of the recombinant phage from each individual clone is analyzed by phage-ELISA, using Raji cell surface antigen extract, as described above. Alternatively, phage-ELISA may be carried out using soluble human CD20 antigen. In a 96-well ELISA plate, 50 μl of carbonate coating buffer, pH 9.6, containing 50 ng of soluble human CD20 antigen is added. After overnight incubation at 4° C., the wells are washed 3 times with 200 μl of borate washing buffer, pH 8.0, and then blocked with 200 μl of the same buffer at 37° C. for 1 hr. After blocking, the wells are washed 3 times with 200 μl of borate washing buffer and 100 μl of scFv-phage containing supernatant is added to each well which is then incubated at 37° C. for 1 hr.

After incubation, the wells are washed 5 times with 200 μl of borate washing buffer, pH 8.0 and then incubated with 100 μl of 5,000 fold-diluted (in borate washing buffer) horseradish peroxidase conjugated anti-M13 mouse antibody (HRP/anti-M13 mouse Ab, Amersham). After one hour incubation at 37° C. and 3-time washing with 200 μl of borate washing buffer, 100 μl of o-phenylenediamine (OPD, from Sigma) substrate solution is added for color development.

Substrate solution is prepared by dissolving 10 mg of OPD in 10 ml of citric phosphate buffer, pH 5.0 (24 mM citric acid (Sigma), 51 mM Na₂HPO₄ (Sigma)), with 8 μl of 30% H₂O₂ (BDH). After color development at room temperature for 1 hr, the reaction is stopped by adding 100 μl of 40% H₂SO₄ (Sigma). The color intensity is measured at absorbance 450 nm with a Sunrise micro-plate reader (Tecan). Potential phage candidates are identified by selecting those with ELISA reading 1.5 fold more than the mean value of the sample set. The isolated ones are later subjected to further analysis by phage-ELISA in the presence of control antigens (BSA) and nucleotide sequence determination.

By performing rounds of panning, re-engineered antibodies containing human CDRs that are detrimental to the resultant immunoreactivities are screened out, and only those retaining immunoreactivities will be isolated and used for further analyses.

10. Conversion of Leads from a Positive Phage Library into a Functional Antibody

The DNA sequences of the scFv in scFv-Phages that are positive for CD20 antigen are elucidated. The VH and VL sequences of the selected scFv-Phages are PCR-amplified using sequence specific-primers with the appropriate cloning sites incorporated. The VH and VL sequences are then subcloned into their corresponding staging vectors as described in Orlandi et al (1989. Cloning immunoglobulin variable domains for expression by the polymerase chain reaction. PNAS 3833-3837). The respective heavy and light chain variable regions are subcloned into final expression vectors similar to the pSV-gpt and pSV-hyg containing human IgG and human kappa sequences, as described by Orlandi et al (1989).

The heavy and light chain expression vectors are transfected into Sp2/0 mouse myeloma cells by electroporation using conditions as described by Co et al. (1992. Chimeric and humanized antibodies with specificity for the CD33 antigen. J Immunol. 148:1149-1154). and cells selected for hygromycin expression. Clones secreting a maximal amount of complete antibody are detected by ELISA. Purified antibody is used to test for binding to the CD20 antigen. Briefly, into triplicate wells of ELISA plate coated with CD20 antigen are added with varying concentrations of the purified antibodies (from 0.01-10 μg/ml). The plates are incubated for 1 hr at room temperature. Unbound antibodies are removed by washing three times with buffer (PBS containing 0.05% polysorbate-20). Horseradish peroxidase (HRP)-conjugated goat anti-human IgG, Fc fragment-specific antibodies (Jackson ImmunoResearch) are added to the wells (100 μl of antibody stock diluted ×10⁴). Following incubation for 1 hr, the plates are washed three times. A reaction solution (100 μl, containing 167 μg of o-phenylenediamine[OPD; Sigma, St. Louis, Mo.], 0.025% hydrogen peroxide in PBS) is added to the wells. Color is allowed to develop in the dark for 30 min. The reaction is stopped by the addition of 50 μl of 4 N HCl solution into each well before measuring absorbance at 490 nm in an automated ELISA reader.

11. Comparing the Binding Affinity of the Various CDR-Humanized Anti-CD20 Antibodies.

Antibodies carrying different human CDRs and demonstrated to retain immunoreactivities are used to compare with the parent antibody (framework-re-engineered 1F5) for binding to Raji Burkit lymphoma cells in a competitive flow cytometry assay. Briefly, 1 μg of murine 1F5 is mixed with varying concentrations of the CDR-humanized and the parent framework-re-engineered antibodies in a final volume of 100 μl of PBS supplemented with 1% FCS and 0.01% (w/v) sodium azide (PBS-FA). The mixture is incubated for 30 min at 4° C. and washed three times with PBS to remove unbound antibodies. The binding levels of the murine 1F5 onto Raji cells in the presence of varying concentrations of competitors (all containing human Fc portions) are assessed by the addition of a 20× diluted FITC-labeled, goat anti-mouse IgG, Fc fragment-specific antibodies (Jackson ImmunoResearch, West Grove, Pa.) in a final volume of 100 μl in PBS-FA, and incubating for 30 min at 4° C. The mixture is washed three times with PBS and fluorescence intensities are measured by a FACSCAN fluorescence-activated cell sorter (Becton Dickinson, Bedford, Mass.).

### INDUSTRIAL APPLICABILITY

The present invention relates to functionally humanized immunoglobulins having improved immunogenicity and methods of making same. The products and processes of the present invention find utility in the production both diagnostic and therapeutic antibodies.

All patents and publications mentioned herein are incorporated by reference in their entirety. Nothing herein is to be construed as an admission that the invention is not entitled to antedate such disclosure by virtue of prior invention.

While the invention has been described in detail and with reference to specific embodiments thereof, it is to be understood that the foregoing description is exemplary and explanatory in nature and is intended to illustrate the invention and its preferred embodiments. Through routine experimentation, one skilled in the art will readily recognize that various changes and modifications can be made therein without departing from the spirit and scope of the invention. Other advantages and features will become apparent from the claims filed hereafter, with the scope of such claims to be determined by their reasonable equivalents, as would be understood by those skilled in the art. Thus, the invention is intended to be defined not by the above description, but by the following claims and their equivalents.

1. A re-engineered immunoglobulin comprising a parent immunoglobulin having affinity for a target antigen, wherein the amino acid sequence of at least one complementarity-determining region (CDR) of said parent antigen is replaced with an amino acid sequence of a corresponding CDR of a primate immunoglobulin, wherein said re-engineered immunoglobulin binds to the target antigen with an affinity within 50-fold of the affinity of the parent immunoglobulin for said antigen.

2. The re-engineered immunoglobulin of claim 1, wherein the primate CDR has an amino acid sequence that is at least 50% identical to the amino acid sequence of the replaced parent CDR; the primate CDR contains at least one identical aromatic amino acid residue at the corresponding position of said parent CDR; the primate CDR contains at least one identical charged amino acid residue at the corresponding position of said parent CDR; or the primate CDR contains at least one amino acid residue identical to said parent CDR at a position that is determined by crystal structure and/or computer analysis to contribute to maintaining the binding affinity of the re-engineered immunoglobulin within 50-fold of the affinity of the parent immunoglobulin for said antigen.

3. The re-engineered immunoglobulin of claim 2, wherein the primate CDR has an amino acid sequence that is at least 50% identical to the amino acid sequence of the replaced parent CDR; the primate CDR contains at least one identical aromatic amino acid residue at the corresponding position of said parent CDR; the primate CDR contains at least one identical charged amino acid residue at the corresponding position of said parent CDR; and the primate CDR contains at least one amino acid residue identical to said parent CDR at a position that is determined by crystal structure and/or computer analysis to contribute to maintaining the binding affinity of the re-engineered immunoglobulin within 50-fold of the affinity of the parent immunoglobulin for said antigen.

4. The re-engineered immunoglobulin of claim 1, wherein the primate CDR contains at least one conservatively similar aromatic amino acid residue at the corresponding position of said parent CDR; the primate CDR contains at least one conservatively similar charged amino acid residue at the corresponding position of said parent CDR; or the primate CDR contains at least one amino acid residue conservatively similar to said parent CDR at a position that is determined by crystal structure and/or computer analysis to contribute to maintaining the binding affinity of the re-engineered immunoglobulin within 50-fold of the affinity of the parent immunoglobulin for said antigen.

5. The re-engineered immunoglobulin of claim 4, wherein the primate CDR contains at least one conservatively similar aromatic amino acid residue at the corresponding position of said parent CDR; the primate CDR contains at least one conservatively similar charged amino acid residue at the corresponding position of said parent CDR; and the primate CDR contains at least one amino acid residue conservatively similar to said parent CDR at a position that is determined by crystal structure and/or computer analysis to contribute to maintaining the binding affinity of the re-engineered immunoglobulin within 50-fold of the affinity of the parent immunoglobulin for said antigen.

6. The re-engineered immunoglobulin of claim 1, wherein the binding affinity of the re-engineered immunoglobulin is within 10-fold of the affinity of the parent immunoglobulin for said antigen.

7. The re-engineered immunoglobulin of claim 6, wherein the binding affinity of the re-engineered immunoglobulin is within 3-fold of the affinity of the parent immunoglobulin for said antigen

8. The re-engineered immunoglobulin of claim 7, wherein the amino acid sequence of CDR3 of the parent immunoglobulin heavy chain is replaced.

9. The re-engineered immunoglobulin of claim 7, wherein the amino acid sequence of CDR3 of the parent immunoglobulin light chain is replaced.

10. The re-engineered immunoglobulin of claim 7, wherein the amino acid sequences of CDR3 of the parent immunoglobulin light and heavy chains were replaced.

11. The re-engineered immunoglobulin of claim 1, wherein the primate is human.

12. A method of preparing a re-engineered immunoglobulin, the method comprising the steps of:
a. providing an amino acid sequence of a non-human parent immunoglobulin that binds to a target antigen;
b. identifying at least one primate CDR whose amino acid sequence is homologous to the amino acid sequence of a CDR of the parent immunoglobulin;
c. replacing said parent CDR with said primate CDR in said parent immunoglobulin amino acid sequence;
d. preparing a nucleic acid sequence that encodes the amino acid sequence obtained in step c; and
e. expressing the nucleic acid sequence obtained in step d in a recombinant cell to obtain the re-engineered immunoglobulin, wherein said re-engineered immunoglobulin binds to said antigen with an affinity within 50-fold of the affinity of the parent immunoglobulin for said antigen.

13. The method of claim 12, wherein the primate CDR identified in step b has an amino acid sequence that is at least 50% identical to the amino acid sequence of the replaced parent CDR; the primate CDR contains at least one identical aromatic amino acid residue at the corresponding position of said parent CDR; the primate CDR contains at least one identical charged amino acid residue at the corresponding position of said parent CDR; or the primate CDR contains at least one amino acid residue identical to said parent CDR at a position that is determined by crystal structure and/or computer analysis to contribute to maintaining the binding affinity of the re-engineered immunoglobulin within 50-fold of the affinity of the parent immunoglobulin for said antigen.

14. The method of claim 13, wherein the primate CDR identified in step b contains at least one conservatively similar aromatic amino acid residue at the corresponding position of said parent CDR; the primate CDR contains at least one conservatively similar charged amino acid residue at the corresponding position of said parent CDR; or the primate CDR contains at least one amino acid residue conservatively similar to said parent CDR at a position that is determined by crystal structure and/or computer analysis to contribute to maintaining the binding affinity of the re-engineered immunoglobulin within 50-fold of the affinity of the parent immunoglobulin for said antigen.

15. The method of claim 12, wherein the primate CDR identified in step b contains at least one conservatively similar aromatic amino acid residue at the corresponding position of said parent CDR; the primate CDR contains at least one conservatively similar charged amino acid residue at the corresponding position of said parent CDR; or the primate CDR contains at least one amino acid residue conservatively similar to said parent CDR at a position that is determined by crystal structure and/or computer analysis to contribute to maintaining the binding affinity of the re-engineered immunoglobulin within 50-fold of the affinity of the parent immunoglobulin for said antigen.

16. The method of claim 15, wherein the primate CDR identified in step b contains at least one conservatively similar aromatic amino acid residue at the corresponding position of said parent CDR; the primate CDR contains at least one conservatively similar charged amino acid residue at the corresponding position of said parent CDR; and the primate CDR contains at least one amino acid residue conservatively similar to said parent CDR at a position that is determined by crystal structure and/or computer analysis to contribute to maintaining the binding affinity of the re-engineered immunoglobulin within 50-fold of the affinity of the parent immunoglobulin for said antigen.
